SEQUENCE LISTING (17 sequences)

SEQ ID NO: 1 - DNA, 18146 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatgttccgctaattcaacccattgcggtcccaaaagggtcagtgctgcaacattttgctgccggtcactcgcgcgcattatgctgagtgatatcccgcttaacctcgagcctagggatatgtcaacttcagccttcaaatgtatgtgaattcaacctcagtaattttgagcaaaaagttgatgaggtgtttattcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccaaagaacaattgtttgttatcaaaaccgttcagtcaatcctgtagatgaaacacgtactgtgttcagtaaaaaggttgttaacaaatgtctgtctaataaagtgaatattaagtgacatagtgttaaggtcacccagtcttcaaatgtatgtgattcaactgacacggaaatttgtcgaaccttttaaggtgaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgaccttttactacagtaccgaaatcttcgatctagatctcagctgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactggtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctactcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgatggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaccgtcatgtagatgcataatcagtagcgataatggtaccagctccactcggggtgcaagacgaagtgagaggggtagaggggggggaggggtgggggttaaaacataaataaataaaaaattaataaaacacgtcgctacccccgccccccccccccccccgcgcgcggtccgccccgccccgccccgcaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgtccccgccccgccccgctccgcctctccacgccgccgtcggttagtctcgccgcgcgaggctttcaaaggaaaataccgctccgccgccgccgccgccgggatatttttcgcttcgcgcgccgcccgcccctcagcgacgctgcgacggaagcggggcacggggcgaggcggcggcggagcgcggcgggcccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtggggccgagactgactggcgcaatgagggtgtccactcgcccgccctgccgggaagaggaggcccgacattaatcgcgaaccaaattactgccgaacaaagaaaagacaccgacgcactttcggaactccccgaggccctcccgggaaacacgcccccctcgccgagccccccacgcacgcacacacacagcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcaggcgcacccctcgcggcgcacgccgaggcgcgacgggccgccgacactcgcgacgcccgcgccgcgccccgaaacacgcgaggcgtcacacgcgctcccctcgcgccggcccccgccacggggcgccacgccccccccgacgctccccttgtttccgacgcacgccccacacacgcacccccccactcgtccgggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcggggcccacacccgcgcagccagcccgacgttgggggggacgtggggggaggggctcaacgactcgtgccgggccgaagcccacgccccgaggcatgccccgcaccgcgccccgagcggcacggcccgccccccaccgccgtccacccccacggcccgccccgccccggcggagcccggcccctcccgagccccgaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcactccccgcgccgccgggggcctcgcggccgccgacagctccgcgccgctcggcgtcggtaacggaaaataccattagcacgctctcccgcgtccctgaaggaaacagggtttagacacgcctcggctttagaccctccgcggcggcgtgggggagatcgcccgcgccccgcttcgccacgccgcggccgtggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttccttcctttacccgcccctcccggaagcacgcagcggcgcggcggcaggggaagagggagaggtcggagccccgacaggcgcccccctgccgacggaagccccccctgccccgtcccgccccaagccgaagaccgcacactggccgccgagatctcggagacgattggtacaagtacggaagaagaaaaacctacagctcctgggcaacgtgctggttattgtgctgtctcatcattttggcaaagaattctgcagtcgacggtaccgcgggcccgggatccgccaccatgtggaccctggtgtcttgggtggccctgacagctggactggtggccggcacaagatgccccgatggccagttttgccctgtggcctgctgggatgtcgaggacccgttgcacgaccaataacacgacagagtagtaaaaccgtttcttaagacgtcagctgccatggcgcccgggccctaggcggtggtacacctgggaccacagaacccaccgggactgtcgacctgaccaccggccgtgttctacggggctaccggtcaaaacgggacaccggacgactctggacccaggcggagccagctacagctgctgcagacccctgctggacaagtggcctaccaccctgagcagacacctgggcggaccttgtcaggtggacgcccactgttctgccggacacagctgcatcttcaccgtgtctggcacctccagctgctgtccattccctgaggctgtggcttgtggcgacagacctgggtccgcctcggtcgatgtcgacgacgtctggggacgacctgttcaccggatggtgggactcgtctgtggacccgcctggaacagtccacctgcgggtgacaagacggcctgtgtcgacgtagaagtggcacagaccgtggaggtcgacgacaggtaagggactccgacaccgaacaccgctgggccaccactgttgccctagaggctttcactgcagcgccgacggcagaagctgcttccagagaagcggcaacaacagcgtgggcgccatccagtgcccagacagccagttcgagtgccccgacttcagcacctgttgcgtgatggtggatggcagctggggctgctgccctatgcctcaggctagctgctccggtggtgacaacgggatctccgaaagtgacgtcgcggctgccgtcttcgacgaaggtctcttcgccgttgttgtcgcacccgcggtaggtcacgggtctgtcggtcaagctcacggggctgaagtcgtggacaacgcactaccacctaccgtcgaccccgacgacgggatacggagtccgatcgacgagcgaggacagagtgcactgttgtccccacggcgccttttgcgacctggtgcacaccagatgcatcacccctaccggcacacaccccctggccaagaaactgcccgcccagaggaccaatagagccgtggccctgtccagctccgtgatgtgccctgacgccagatccagatgtcccgacggctccacatgcgctcctgtctcacgtgacaacaggggtgccgcggaaaacgctggaccacgtgtggtctacgtagtggggatggccgtgtgtgggggaccggttctttgacgggcgggtctcctggttatctcggcaccgggacaggtcgaggcactacacgggactgcggtctaggtctacagggctgccgaggtgtacctgcgaactgcccagcggcaaatacggctgttgccccatgcccaacgccacctgttgtagcgaccatctgcactgctgcccacaagacaccgtgtgcgacctgatccagagcaagtgcctgagcaaagagaacgccaccaccgacctgctgaccaagctgcctgctcataccgtgggcgacgtgaagtgcgacgcttgacgggtcgccgtttatgccgacaacggggtacgggttgcggtggacaacatcgctggtagacgtgacgacgggtgttctgtggcacacgctggactaggtctcgttcacggactcgtttctcttgcggtggtggctggacgactggttcgacggacgagtatggcacccgctgcacttcacggacatggaagtgtcctgcccagacggctatacctgctgcaggctgcagtctggcgcctggggctgttgtccttttacccaggccgtgtgttgcgaggaccacatccattgctgccctgccggcttcacctgtgacacccagaagggaacctgcgagcagggccctcatcaggtgccctggatggaaaaagctgtaccttcacaggacgggtctgccgatatggacgacgtccgacgtcagaccgcggaccccgacaacaggaaaatgggtccggcacacaacgctcctggtgtaggtaacgacgggacggccgaagtggacactgtgggtcttcccttggacgctcgtcccgggagtagtccacgggacctacctttttccccctgcccacctgagcctgcctgatcctcaggccctgaagagggacgtgccctgcgacaatgtgtccagctgccccagcagcgatacctgttgccagctgacatctggcgagtggggatgctgtcctatccccgaagccgtgtgctgctccgaccaccagcattgctgtccacaaggctacacctgtgtggggacgggtggactcggacggactaggagtccgggacttctccctgcacgggacgctgttacacaggtcgacggggtcgtcgctatggacaacggtcgactgtagaccgctcacccctacgacaggataggggcttcggcacacgacgaggctggtggtcgtaacgacaggtgttccgatgtggacacaggccgagggccagtgtcagaggggctctgaaattgtggccggcctggaaaagatgcccgccagaagggccagcctgagccaccctagagacatcggctgcgaccagcacaccagctgtcctgtgggccagacatgctgtccaagcctgggaggctcttgggcttgctgccagctgcctcacgctgtgtgcccggctcccggtcacagtctccccgagactttaacaccggccggaccttttctacgggcggtcttcccggtcggactcggtgggatctctgtagccgacgctggtcgtgtggtcgacaggacacccggtctgtacgacaggttcggaccctccgagaacccgaacgacggtcgacggagtgcgacacacgtgcgaagatcggcagcactgctgtcccgccggatacacctgtaacgtgaaggcccggtcctgcgagaaagaagtggtgtctgcccagcccgccaccttcctggccagatcacctcacgtgggcgtgaaggacgtggaatgcggcgagggccacttctgccacgacaaccagacctgctgccgggacaataacgcttctagccgtcgtgacgacagggcggcctatgtggacattgcacttccgggccaggacgctctttcttcaccacagacgggtcgggcggtggaaggaccggtctagtggagtgcacccgcacttcctgcaccttacgccgctcccggtgaagacggtgctgttggtctggacgacggccctgttatgacagggctgggcctgttgcccatacagacagggcgtgtgctgtgccgacaggcggcactgttgcccagccggctttagatgtgccgccagaggcaccaagtgtctgcggagagaagcccccagatgggatgcccccctgagagatcctgccctgagacagctgctgtgactcgagtctagagggcccgtctgtcccgacccggacaacgggtatgtctgtcccgcacacgacacggctgtccgccgtgacaacgggtcggccgaaatctacacggcggtctccgtggttcacagacgcctctcttcgggggtctaccctacggggggactctctaggacgggactctgtcgacgacactgagctcagatctcccgggcattaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaatttgggcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagttccccctcctaacccttctgttatcgtccgtacgacccctacgccacccgagataccgaagactccgcctttcttggtcttaagacaccttacacacagtcaatcccacacctttcaggggtccgaggggtccgtccgtcttcatacgtttcgtacgtagagttaatcagtcgttggtccacacctttctccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccaggggtccgaggggtcgtccgtcttcatacgtttcgtacgtagagttaatcagtcgttggtatcagggcggggattgaggcgggtagggcggggattgaggcgggtcaaggcgggtaagaggcggggtaccgactgattaaaaaaaataaatacgtctccggctccggcggagacggagactcgataaggagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttcttcatcactcctccgaaaaaacctccggatccgaaaacgtttttcgagggccctcgaacatataggtaaaagcctagactagtcgtgcacaactgttaattagtagccgtatcatatagccgtatcatattatgctgttccactccttgatttggtaccactaacttgttctacctaacgtgcgtccatctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacggagaggccggcgaacccacctctccgataagccgatactgacccgtgttgtctgttagccgacgagactacggcggcacaaggccgacagtcgcgtccccgcgggccaagaaaaacagttctggctggacaggccacgggacttacttgacgtcctgctccgtcgcgccgatagcaccgaccggtgctgccgcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagccgcaaggaacgcgtcgacacgagctgcaacagtgacttcgcccttccctgaccgacgataacccgcttcacggccccgtcctagaggacagtagagtggaacgaggacggctctttcataggtagtaccgactacgttacgccgccgacgtatgcgaactaggccgatggacgggtaagctggtggttcggaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatctttgtagcgtagctcgctcgtgcatgagcctaccttcggccagaacagctagtcctactagacctgcttctcgtagtccccgagcgcggtcggcttgacaagcggtccgagttccgcgcgtacgggctgccgctcctagagcagcactgggtaccgctacggacgaacggcttatagtaccaccttttaggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttctccggcgaaaagacctaagtagctgacaccggccgacccacaccgcctggcgatagtcctgtatcgcaaccgatgggcactataacgacttctcgaaccgccgcttacccgactggcgaaggagcacgaaatgccatagcggcgagggctaagcgtcgcgtagcggaagatagcggaagaactgctcaagatctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattagactcgccctgagaccccaagctttactggctggttcgctgcgggttggacggtagtgctctaaagctaaggtggcggcggaagatactttccaacccgaagccttagcaaaaggccctgcggccgacctactaggaggtcgcgcccctagagtacgacctcaagaagcgggtggggttgaacaaataagcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacaccccgtcgaatattaccaatgtttatttcgttatcgtagtgtttaaagtgtttatttcgtaaaaaaagtgacgtaagatcaacaccaaacaggtttgagtagttacatagaatagtacagacatctaggcgcataccacgtgagagtcatgttagacgagactacggcgtatcaattcggtcggggctgtggggccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgcggttgtgggcgactgcgcgggactgcccgaacagacgagggccgtaggcgaatgtctgttcgacactggcagaggcccttagttcgaacaccttccgatgagctttacaaactgggttcaatttgttaaatttccgttacgatggtttatgattaactcacatacatttgaagactgggtgacccttactgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaaactactttctttattttcgactttacttagtaagagagatgataataagactataaagtgtaagaattttatttcaccactaggattgactggattctgtcccttaaaaatgatcctaatttacagtccttaacactttttcactcaaatttacataaaccgattccacatacatttgaaggctgaagttctgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgacatatccctaggagatcgatctcagctggagctcccccccgggccatgggtcgaaaacaagggaaatcactcccaattaaagctcgaaccgcattagtaccagtatcgacaaaggacacactttaacaataggcgagtgttaaggtgtgttgtatgctcggccttcgtatttcacatttcggaccccagcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggcggattactcactcgattgagtgtaattaacgcaacgcgagtgacgggcgaaaggtcagccctttggacagcacggtcgacgtaattacttagccggttgcgcgcccctctccgccaaacgcataacccgcgagaaggcgaaggagcgagtgactgagcgacgcgagccagcaagccgacgccgctcgcctatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaaatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttcccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtggggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacaccacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacaggtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtctatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgctaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggtgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacgggggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgtcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcatgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtcatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttccgaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcgcgggacatcgccgcgtaattccgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagcccccgagggaaatcccaaggctaaatcacgaaatgccgtggagctggggttttttgaactatagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacggctaaagccggataaccaattttttactcgacttttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgaaattgtttttaaattgcgcttaaaattgttttataattgcgaatgttaaaggtaagcggtaagtccgacgcgttgacaacccttcccgctagccacgcccggagaagcgataatgcggtcgaccgctttccccctacacgac

SEQ ID NO: 2 - protein, 526 aa, Homo sapiens
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - DNA, 9109 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgtacgccctgtttctgctggccagcctgctgggcgccgctctggctggacctgttctgggactgaaggagtgcaccaggggcagcgccgtgtggtgtcagaacgtgaagaccgccagcgattgcggcgccgtgaagcactgcctgcagacagtgtggaacaagcctacagtgaagagcctgccttgtgatatctgcaaggatgtggtgaccgccgccggagatatgctgaaggacaacgccaccgaggaggagatcctggtgtacctggagaagacctgcgattggctgcctaagcctaatatgagcgccagctgcaaggagatcgtggacagctacctgcccgtgatcctggacatcatcaagggcgagatgtccagacctggcgaggtgtgcagcgccctgaacctgtgtgagagcctgcagaagcacctggccgagctgaatcaccagaagcagctggagagcaacaagatccctgagctggacatgaccgaggtggtggccccttttatggccaacatccctctgctgctgtaccctcaggatggccctaggagcaagcctcagcccaaggacaatggcgatgtgtgtcaggactgcatccagatggtgacagatatccagacagccgtgaggaccaatagcaccttcgtgcaggccctggtggagcacgtgaaggaggagtgtgataggctgggccccggcatggccgacatctgtaagaattacatcagccagtacagcgagatcgccatccagatgatgatgcacatgcagcctaaggagatctgcgccctggtgggcttttgcgatgaggtgaaggagatgcccatgcagaccctggtgcccgccaaggtggcctctaagaacgtgatccccgccctggagctggtggagcctatcaagaagcacgaggtgcctgccaagtccgacgtgtactgtgaggtgtgcgagttcctggtgaaggaggtgaccaagctgatcgacaataataagaccgagaaggagatcctggacgcctttgacaagatgtgctccaagctgcccaagtccctgagcgaggagtgccaggaggtggtggatacctacggctccagcatcctgtccatcctgctggaggaggtgagccctgagctggtgtgctccatgctgcacctgtgtagcggcaccagactgcctgccctgacagtgcacgtgacccagcctaaggacggcggcttttgtgaggtgtgtaagaagctggtgggctacctggataggaatctggagaagaactccaccaagcaggagatcctggccgccctggagaagggctgcagctttctgcccgatccttaccagaagcagtgtgatcagttcgtggccgagtacgagcctgtgctgatcgagatcctggtcgaggtcatggacccttcctttgtgtgcctgaagatcggcgcctgcccttccgcccacaagcctctgctgggcacagagaagtgcatctggggccccagctactggtgccagaacaccgagacagccgcccagtgtaatgccgtggagcactgcaagaggcacgtgtggaactgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtggggatcgggagatgggggaggctaactgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 4 - protein, 524 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 5 - DNA, 8869 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgtacgccctgtttctgctggccagcctgctgggcgccgctctggctggacctgttctgggactgaaggagtgcaccaggggcagcgccgtgtggtgtcagaacgtgaagaccgccagcgattgcggcgccgtgaagcactgcctgcagacagtgtggaacaagcctacagtgaagagcctgccttgtgatatctgcaaggatgtggtgaccgccgccggagatatgctgaaggacaacgccaccgaggaggagatcctggtgtacctggagaagacctgcgattggctgcctaagcctaatatgagcgccagctgcaaggagatcgtggacagctacctgcccgtgatcctggacatcatcaagggcgagatgtccagacctggcgaggtgtgcagcgccctgaacctgtgtgagagcctgcagaagcacctggccgagctgaatcaccagaagcagctggagagcaacaagatccctgagctggacatgaccgaggtggtggccccttttatggccaacatccctctgctgctgtaccctcaggatggccctaggagcaagcctcagcccaaggacaatggcgatgtgtgtcaggactgcatccagatggtgacagatatccagacagccgtgaggaccaatagcaccttcgtgcaggccctggtggagcacgtgaaggaggagtgtgataggctgggccccggcatggccgacatctgtaagaattacatcagccagtacagcgagatcgccatccagatgatgatgcacatgcagcctaaggagatctgcgccctggtgggcttttgcgatgaggtgaaggagatgcccatgcagaccctggtgcccgccaaggtggcctctaagaacgtgatccccgccctggagctggtggagcctatcaagaagcacgaggtgcctgccaagtccgacgtgtactgtgaggtgtgcgagttcctggtgaaggaggtgaccaagctgatcgacaataataagaccgagaaggagatcctggacgcctttgacaagatgtgctccaagctgcccaagtccctgagcgaggagtgccaggaggtggtggatacctacggctccagcatcctgtccatcctgctggaggaggtgagccctgagctggtgtgctccatgctgcacctgtgtagcggcaccagactgcctgccctgacagtgcacgtgacccagcctaaggacggcggcttttgtgaggtgtgtaagaagctggtgggctacctggataggaatctggagaagaactccaccaagcaggagatcctggccgccctggagaagggctgcagctttctgcccgatccttaccagaagcagtgtgatcagttcgtggccgagtacgagcctgtgctgatcgagatcctggtcgaggtcatggacccttcctttgtgtgcctgaagatcggcgcctgcccttccgcccacaagcctctgctgggcacagagaagtgcatctggggccccagctactggtgccagaacaccgagacagccgcccagtgtaatgccgtggagcactgcaagaggcacgtgtggaactgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 6 - DNA, 8974 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgccgctgctgctactgctgcccctgctgtgggcaggggcgctagctgaagtgcagctgctcgagtccggaggaggactggtgcaacccggcggaagcctcagactgagctgcgccgcttccggctttaccttcagcagctacgccatgagctgggtcagacaagcccccggcaaaggactggagtgggtcagctccatttatggcagcggcggatacacatcctacgctgattccgtcaaaggaagattcaccatcagcagagacaactccaagaacaccctctatctccagatgaactctctgagagccgaagacaccgctgtctactattgcgccagaacctacggaggcagattcgactactggggacaaggcaccctcgtcaccgtctccagcggaggaggaggaagcggaggcggaggatccggaggcggcggaagcgacattcagatgacccagagccctagctccctctccgcttccgtgggagacagagtgaccatcacatgcagagcctcccaatccatcagctcctatctcaactggtaccagcaaaagcccggcaaagctcctaagctcctcatctatgctgcttcctctctgcagagcggcgtgcctagcagattttccggctccggctccggaaccgatttcacactgacaatcagctctctgcagcccgaggacttcgccacctactactgccaacagtatacctacccccccacattcggccaaggaaccaagctcgagatcaagggaggaggcggcagcaaaccttgtatttgcaccggaagcgaggtgtcctccgtctttatcttccctcctaagcccaaggacgtcctcaccattaccctcacacccaaggtgacatgcgtggtggtcgatattagccaagacgaccccgaggtgcacttcagctggttcgtggatgacgtcgaagtgcacaccgcccagacaagaccccccgaggagcagttcaacagcaccttcagaagcgtgtccgagctccccattctgcaccaagactggctcaatggaagaacctttaggtgcaaggtcacctccgccgcctttccctcccccatcgagaagacaatcagcaagcccgagggcagaacccaagtgcctcacgtgtacacaatgagccccaccaaggaggagatgacacagaacgaagtgtccatcacatgcatggtgaagggcttctacccccccgatatctatgtggagtggcagatgaacggccagcctcaagagaactacaaaaacaccccccctaccatggacaccgacggcagctattttctgtacagcaagctgaatgtgaagaaagagaaatggcaacaaggcaacaccttcacatgcagcgtgctgcacgagggcctccacaatcaccacaccgaaaagtctctgtcccacagccccggcaagtgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtggggatcgggagatgggggaggctaactgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 7 - protein, 479 aa, Homo sapiens
MPLLLLLPLLWAGALAEVQLLESGGGLVQPGGSLRLSCAASGFTFSSYAMSWVRQAPGKGLEWVSSIYGSGGYTSYADSVKGRFTISRDNSKNTLYLQMNSLRAEDTAVYYCARTYGGRFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYTYPPTFGQGTKLEIKGGGGSKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 8 - DNA, 8980 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgccgctgctgctactgctgcccctgctgtgggcaggggcgctagctgaagtgcagctgctggagagcggcggaggactggtgcaacccggcggaagcctcagactgagctgcgccgcctccggattcacattctacggcagcggcatgagctgggtcagacaagcccccggaaagggactcgaatgggtgtccggcatcagcagctacggaggcagcacatattacgccgacagcgtgaagggaagattcaccatctctagagacaactccaagaacaccctctatctgcagatgaattctctgagggccgaggacaccgccgtgtattattgcgctagggccaactactggcactccagcctcgattattggggccaaggcacactggtgacagtgagctccggcggaggaggaagcggaggaggaggatccggcggcggaggcagcgatatccagatgacacaaagccccagcagcctctccgcttccgtgggagatagggtgaccattacatgcagagcttcccagagcatctcctcctacctcaactggtatcagcagaaacccggcaaggctcccaagctgctgatctatgctgccagctctctgcagagcggagtgcctagcagattctccggctccggaagcggaaccgacttcacactgacaatcagctccctccagcccgaggacttcgccacctattactgtcagcagagcgctggactgctgaccttcggccaaggcaccaagctggagatcaagggaggcggcggctccaagccttgcatctgcaccggaagcgaggtgagcagcgtgttcattttccctcccaagcccaaggacgtgctcaccatcacactcacacccaaagtgacatgcgtggtggtggatatctcccaagacgaccccgaggtgcacttcagctggttcgtggacgacgtggaagtgcatacagctcagacaagacctcccgaggagcagttcaactccacctttaggtccgtcagcgaactccctatcctccatcaagactggctgaatggaagaacatttagatgcaaggtgaccagcgccgccttcccttcccccatcgagaagacaatcagcaagcccgagggaaggacccaagtgcctcacgtctacacaatgagccccaccaaggaggagatgacacagaacgaggtgagcatcacatgcatggtcaagggcttctatcctcccgatatctacgtcgaatggcagatgaacggccagccccaagaaaactacaagaataccccccccaccatggacacagacggcagctactttctgtacagcaaactgaacgtgaagaaagagaagtggcagcaaggcaacacattcacatgctccgtgctgcacgaaggactgcacaaccaccataccgagaagtccctcagccatagccccggcaagtgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtggggatcgggagatgggggaggctaactgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 9 - protein, 481 aa, Homo sapiens
MPLLLLLPLLWAGALAEVQLLESGGGLVQPGGSLRLSCAASGFTFYGSGMSWVRQAPGKGLEWVSGISSYGGSTYYADSVKGRFTISRDNSKNTLYLQMNSLRAEDTAVYYCARANYWHSSLDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSAGLLTFGQGTKLEIKGGGGSKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 10 - DNA, 8974 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgccgctgctgctactgctgcccctgctgtgggcaggggcgctagctgaagtccagctgctggagtccggaggaggactggtgcaacccggcggatctctgagactcagctgcgccgccagcggcttcacatttagcagctacgccatgagctgggtgagacaagcccccggaaaaggactggagtgggtgagcgccatttccggaagcggcggatccacatattacgccgacagcgtcaaaggaagatttaccatctccagagacaactccaaaaacacactgtatctgcagatgaactccctcagagccgaggacaccgccgtgtactactgcgccagaagctatagcgccttcgactattggggccaaggcacactggtgacagtgagcagcggaggaggcggaagcggaggaggaggatccggaggcggaggaagcgatatccaaatgacacagagccctagctccctcagcgcttccgtgggcgatagggtcacaatcacatgtagggctagccagtccatctcctcctatctgaactggtaccagcagaagcccggcaaagcccccaagctgctgatctacgctgctagctctctgcaaagcggcgtgccctccagattctccggcagcggatccggcaccgacttcacactgacaatcagctccctccagcccgaggactttgccacatactactgccagcagatcaccggctacctcttcacctttggccaaggcacaaagctcgagattaagggaggcggcggcagcaagccttgcatctgtaccggcagcgaggtgagcagcgtgttcattttccctcctaagcccaaggacgtgctgacaatcacactgacacctaaggtcacatgcgtggtcgtggacatctcccaagacgatcccgaagtgcacttcagctggttcgtcgatgacgtcgaggtccataccgcccagacaagaccccccgaggagcagttcaacagcaccttcagaagcgtgtccgagctccccattctgcaccaagactggctcaatggaagaacctttaggtgcaaggtcacctccgctgccttcccttcccccatcgagaagacaatcagcaagcccgagggcagaacccaagtgccccatgtgtataccatgagccccaccaaggaggagatgacccagaacgaggtctccatcacatgcatggtgaagggcttctaccctcccgatatctacgtggaatggcagatgaacggccaacctcaagagaactacaagaacaccccccccacaatggataccgatggcagctactttctgtactccaagctcaacgtgaagaaggagaagtggcagcaaggcaacaccttcacatgctccgtgctgcacgagggactgcataatcaccacaccgagaagtctctgagccactcccccggcaaatgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtggggatcgggagatgggggaggctaactgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 11 - protein, 479 aa, Homo sapiens
MPLLLLLPLLWAGALAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSYSAFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQITGYLFTFGQGTKLEIKGGGGSKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 12 - DNA, 8200 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgggcatcctgccttcccccggcatgcctgccctgctgagcctggtgagcctgctgagcgtgctgctgatgggctgcgtggccctgcctgaggtgcagctgctggagagcggcggcggactggtgcagccaggaggatccctgagactgagctgtgccgcctccggctttaccttcagcagctacgccatgtcctgggtgaggcaggcccccggaaagggcctggagtgggtgagcgccatcagcggcagcggcggatccacctactacgccgacagcgtgaagggcaggttcacaatctccagggacaattccaagaacaccctgtacctgcagatgaatagcctgagggccgaggacaccgccgtgtactactgtgccaggagctacagcgcctttgactactggggccagggcaccctggtgaccgtgagctccggcggaggcggatccggaggaggaggatccggcggaggaggcagcgatatccagatgacccagtccccttcctccctgagcgcctccgtgggcgacagggtgaccatcacctgcagagccagccagtccatcagctcctacctgaactggtaccagcagaagcctggcaaggcccccaagctgctgatctacgccgccagctccctgcagagcggcgtgcctagcaggttttccggcagcggctccggcaccgattttacactgacaatcagctccctgcaacccgaggattttgccacatactactgtcagcagatcaccggctacctgttcaccttcggccagggcacaaagctggagatcaagaggacagactacaaggatcacgacggcgattacaaggatcatgacatcgactacaaggacgatgacgataaggccgccgcccaccaccaccaccatcactgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 13 - protein, 301 aa, Homo sapiens
MGILPSPGMPALLSLVSLLSVLLMGCVALPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSYSAFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQITGYLFTFGQGTKLEIKRTDYKDHDGDYKDHDIDYKDDDDKAAAHHHHHH

SEQ ID NO: 14 - DNA, 8127 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgggcatcctgcccagccctggcatgcccgctctgctgagcctggtgagcctgctgagcgtgctgctgatgggctgcgtggccctgcccgaggtgcagctgctggagagcggcggaggcctggtgcagccaggaggatccctgaggctgagctgcgccgccagcggattcacattcagctcctacgccatgagctgggtgagacaggcccccggcaagggcctggagtgggtttcctccatctacggctccggcggctacacctcctacgccgactccgtgaagggcaggtttaccatcagcagggataatagcaagaatacactgtacctgcagatgaattccctgagagccgaggataccgccgtgtactactgcgccaggacctacggcggcaggttcgattactggggccagggcacactggtgacagtgtcctccggcggcggcggatccggaggaggaggatctggcggcggcggttccgatatccagatgacccagtccccttcctccctgagcgcctccgtgggcgatagagtgaccatcacatgtagagcctcccagagcatcagctcctacctgaactggtaccagcagaagcccggcaaggcccccaagctgctgatctacgccgcctcctccctgcagtccggcgtgccttccaggttctccggctccggctccggaacagattttacactgaccatctcctccctgcaacccgaggactttgccacatactactgtcagcagtacacctaccctcctacctttggccagggcaccaagctggagatcaagaggacagattacaaggaccacgatggcgactacaaggatcacgacatcgactacaaggacgatgacgacaaggccgccgcccaccaccaccaccatcactgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 15 - protein, 301 aa, Homo sapiens
MGILPSPGMPALLSLVSLLSVLLMGCVALPEVQLLESGGGLVQPGGSLRLSCAASGFTFSSYAMSWVRQAPGKGLEWVSSIYGSGGYTSYADSVKGRFTISRDNSKNTLYLQMNSLRAEDTAVYYCARTYGGRFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYTYPPTFGQGTKLEIKRTDYKDHDGDYKDHDIDYKDDDDKAAAHHHHHH

SEQ ID NO: 16 - DNA, 8206 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgggcatcctgcctagccccggcatgcccgctctgctgtccctggtgagcctgctgtccgtgctgctgatgggctgcgtggccctgcctgaggtgcagctgctggagagcggcggcggactggtgcagcctggaggaagcctgaggctgtcctgcgccgccagcggattcacattttacggctccggcatgtcctgggtgagacaggcccctggcaagggcctggagtgggtgagcggcatcagctcctacggcggctccacctactacgccgactccgtgaagggcagattcactatctccagggataattccaagaataccctgtacctgcagatgaactccctgagggccgaggacacagccgtgtactactgcgccagagccaactactggcacagcagcctggactactggggccagggcaccctggtgaccgtgagcagcggcggaggcggaagcggaggaggaggatccggcggaggcggtagcgatatccagatgacccagagccctagctccctgagcgccagcgtgggcgatagagtgacaatcacctgcagagcctcccagagcatcagcagctacctgaactggtaccagcagaagcctggcaaggcccccaagctgctgatctacgccgccagcagcctgcagagcggcgtgccttccaggttttccggcagcggcagcggcaccgatttcacactgaccatcagctccctgcagcctgaggatttcgccacatactactgccagcagagcgccggcctgctgaccttcggccagggaaccaagctggagatcaagagaacagactacaaggatcacgacggcgactacaaggaccacgatatcgattacaaggatgatgacgacaaggccgccgcccaccaccaccaccatcactgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 17 - protein, 303 aa, Homo sapiens
MGILPSPGMPALLSLVSLLSVLLMGCVALPEVQLLESGGGLVQPGGSLRLSCAASGFTFYGSGMSWVRQAPGKGLEWVSGISSYGGSTYYADSVKGRFTISRDNSKNTLYLQMNSLRAEDTAVYYCARANYWHSSLDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSAGLLTFGQGTKLEIKRTDYKDHDGDYKDHDIDYKDDDDKAAAHHHHHH

(19) United States
(12) Patent Application Publication
(10) Pub. No.: US 2023/0257436 A1
(43) Pub. Date: Aug. 17, 2023

(54) Cell Lines Secreting Alpha-Synuclein Targeting Antibodies, Progranulin and Prosaposin and a Complex of Both, and GDNF

(71) Applicants: Johan Gunnarsson LUNDKVIST, Huddinge (SE); Lars Ulrik WAHLBERG, Bristol, RI (US); Johan Henrik Biverstål, Stockholm (SE)

(72) Inventors: Johan Gunnarsson LUNDKVIST, Huddinge (SE); Lars Ulrik WAHLBERG, Bristol, RI (US); Johan Henrik Biverstål, Stockholm (SE)

(73) Assignee: Alan F. Feeney Esq., Providence, RI (US)

(21) Appl. No.: 18/011,647

(22) PCT Filed: Jun. 21, 2021
(86) PCT No.: PCT/US21/38312

§ 371 Date: Dec. 20, 2022

Related U.S. Application Data
(60) Provisional application No. 63/212,721, filed on Jun. 20, 2021.
(60) Provisional application No. 63/040,925, filed on Jun. 18, 2020.

(51) Int. Cl. C07K 14/475; G01N 33/68; C12P 21/00; C07K 14/47; A61K 39/395; A61P 25/28

(52) U.S. Cl. CPC C07K 14/4756; G01N 33/6896; C12P 21/005; C07K 14/475; C07K 14/4705; A61K 39/3955; A61P 25/28; G01N 2800/2814; G01N 2333/4756; C07K 2319/00

(57) ABSTRACT

A cell culture comprising a mammalian cell line which is modified to express a heterodimer consisting of a progranulin polypeptide and a prosaposin polypeptide.

### STATEMENT AS TO THE RIGHTS TO INVENTIONS MADE UNDER FEDERALLY SPONSORED RESEARCH AND DEVELOPMENT

This invention was not made with U.S. government support.

### STATEMENT ABOUT THE SEQUENCE LISTING THAT FORMS PART OF THE APPLICATION

The instant application contains a Sequence Listing which has been submitted electronically in ASCII format and is hereby incorporated by reference in its entirety. Said ASCII copy, created on May 15, 2020, is named P9590US00_ST25.txt and is 144,484 bytes in size.

### FIELD OF INVENTION

The present invention concerns methods and compositions for novel biomarkers and treatments including, but not limited to, recombinant proteins and gene- and cell-based therapies, in particular, combinatorial therapies for delivery of progranulin, prosaposin, a complex of progranulin and prosaposin (also referred to herein as “progranulin/prosaposin complex(es)”), alpha-synuclein targeting antibodies and their neurorestorative factors, including, but not limited to, the neurorestorative factor GDNF, in different combinations, for the treatment of neurodegenerative diseases and lysosomal storage disorders. In another aspect, the invention relates to cell lines expressing alpha-synuclein targeting antibodies, GDNF, progranulin, prosaposin, a complex of progranulin and prosaposin, methods of manufacture of such, methods of monitoring such in, e.g., human serum and CSF, and the use of both recombinant factors as therapeutics or a cell line inside of an implantable cell device for the delivery of the alpha-synuclein targeting antibodies, GDNF, progranulin, prosaposin and progranulin/prosaposin complex to a patient.

### BACKGROUND OF THE INVENTION

Frontotemporal dementia (FTD) is a neurological disorder characterized by the atrophy of the frontal lobe and/or anterior temporal lobe as visualized by structural magnetic resonance imaging or positron emission tomography. FTD represents an estimated 10%-20% of all dementia cases. It is recognized as one of the most common presenile dementias, affecting between 15-22 per 100,000 people. Signs and symptoms typically manifest in late adulthood, commonly between the ages of 45 and 65. Signs and symptoms typically include one or more changes in social behavior and conduct, loss of social awareness and poor impulse control, impaired verbal comprehension, progressive, non-fluent aphasia, and marked changes in behavior. As the disease progresses, patients may present symptoms comparable to Alzheimer's Disease, such as loss of executive functioning and working memory. Currently, there is no cure for FTD aside from treatments to manage behavioral symptoms, typical selective serotonin reuptake inhibitors.

One mechanism of FTD is mutations in the granulin (GRN) gene. Haploinsufficiency in GRN readily monitored as decreased extracellular levels of progranulin (PGRN), the precursor form of granulin, typically results in an inheritable form of FTD, and complete loss of PGRN, also leads to a lysosomal storage disorder, neuronal ceroid lipofuscinosis (NCL). Extracellular PGRN is taken up by neurons and transported to the lysosomes via different mechanisms. PGRN also facilitates neuronal uptake and lysosomal delivery of prosaposin (PSAP), the precursor of saposin peptides that are essential for lysosomal glycosphingolipid degradation. Additionally, PGRN mutant neurons have reduced lysosomal GCase activity, lipid accumulation and increased insoluble alpha-synuclein. Brain tissue samples from patients with FTD show reduced levels of PSAP in neurons. Decreased cellular uptake of extracellular PGRN and reduced PGRN-mediated PSAP lysosomal trafficking may therefore be an underlying disease mechanism for NCL and FTD due to GRN mutations. To this end, no one has monitored or characterized the PGRN/PSAP complex in plasma or CSF, and to what extent its expression levels may be altered in disease. There is a need in the art for specific assays to determine the absolute and relative levels of PGRN, PSAP and/or PGRN/PSAP as individual fluid biomarkers and pathway biomarker profiles, respectively, for diagnosis, prognosis, therapy development and to monitor treatment responses.

In addition to regulating each other's expression levels, PGRN and PSAP interact physically to facilitate each other's lysosomal trafficking and PGRN-PSAP interaction is important for maintaining proper lysosomal function in the brain. To date, however, no one has shown that supplementation by either extracellular PGRN or PGRN-PSAP complexes can prevent or treat NCL and FTD. There is a need in the art for an effective method to produce PGRN-PSAP complexes in a way that also allows transport of these molecular entities to the brain where they can protect against neurodegeneration.

### BRIEF SUMMARY OF THE INVENTION

In a first aspect, the invention relates to cell lines which express one or several alpha-synuclein targeting antibodies or antibody fragments, and/or progranulin, and/or prosaposin, and/or GDNF and their subpeptides and derivatives. In a preferred embodiment, the cell line is genetically modified to produce these factors simultaneously, for example, by the insertion of plasmids into the cell line. In various embodiments, the alpha-synuclein targeting antibodies/fragments, GDNF and progranulin and prosaposin are expressed as polypeptides, subpeptides, RNA, or exosomal RNA.

Many different cell types may be encapsulated in the devices according to the present invention. These include well-known, publicly available immortalized cell lines, spontaneously immortalized cell lines as well as dividing primary cell cultures. As cell lines in some embodiments are to be transfected or transduced, clones have to be selected, expanded and cell banked, it is preferable that the cells or cell lines are capable of undergoing a significant number of divisions.

Cell lines with long term propagation potential may be created from a wide variety of cells, including progenitor and/or precursor cells. Also suitable are stem cells including pluripotent and multipotent stem cells, embryonal stem cells, neural stem cells, and hematopoietic stem cells.

Cell lines of the invention include Mouse myeloma cells (NS0), Chinese hamster ovary cells (CHO); CHO-K1; baby hamster kidney cells (BHK); mouse fibroblast-3T3 cells; African green monkey cell lines (including COS-1, COS-7, BSC-1, BSC-40, BMT-10 and Vero); mesenchymal chondroSarcoma-1 (MCS); rat adrenal pheochromocytoma (PC12 and PC12A); AT3, rat glial tumor (C6); rat neuronal cell line RN33b; rat hippocampal cell line HiB5; growth factor expanded stem cells; epidermal growth factor (EGF)-responsive neurospheres; basic fibroblast growth factor-responsive (bFGF-responsive) neural progenitor stem cells derived from the CNS of mammals; foetal cells; primary fibroblasts; Schwann cells; astrocytes; β-TC (ATCC CRL-11506) cells; human liver cancer cell line Hep-G2 striatal cells; oligodendrocytes and their precursors; mouse myoblast cells-C2C12; human glial-derived cells-Hs683; human glial-derived cells-A172; HEI193T cell line; porcine glioblasts; neuronal cells; neurons; astrocytes; interneurons; chondroblasts isolated from human long bone; human embryonic kidney cells 293 (HEK293); human cell line HeLa; rabbit corneal-derived cells (Statens Seruminstitut Rabbit Cornea (SIRC)); Human corneal derived cells, human choroid plexus cells, human induced pluripotent stem cells (iPS) cell derived cell lines, human neurotrophin 3 (NT3) cells, ARPE-19, CAC cells, immortalized human fibroblasts (MDX cells), telomerase immortalized human RPE cell lines such as hTERT RPE-1, mesenchymal stem cells (MSC).

Preferred cell lines for mammalian recombinant production include ARPE-19, CHO, CHO-1, HEI193T, HEK293, COS, NS0, C2C12, and BHK cells.

In a preferred embodiment, the cell line comprises up to four expression constructs; a first expression construct which expresses progranulin polypeptide, a progranulin gene, progranulin RNA or exosomal RNA encoding progranulin and a second expression construct which expresses prosaposin polypeptide, a prosaposin gene, prosaposin RNA or exosomal RNA encoding prosaposin and a third expression construct which expresses a gene, RNA or exosomal RNA encoding an alpha-synuclein antibody or antibody fragment, and a fourth expression construct which expresses GDNF RNA or exosomal RNA encoding GDNF. In an embodiment of the instant invention, the expression constructs comprise plasmids. In a further embodiment, the plasmids may comprise a transposon system such as Sleeping beauty transposase.

Progranulin or Prosaposin produced by cell lines of the invention may further comprise the fragment crystallizable region (Fc region) of an antibody for the purpose of enhancing the distribution and uptake of progranulin, prosaposin and a progranulin/prosaposin complex in the central nervous system. In various embodiments, the prosaposin-Fc or progranulin-Fc region combination comprises a fusion protein, fusion gene or a fusion RNA.

Progranulin and prosaposin expressed by cell lines of the invention typically form a complex, either before or after secretion from the cell line. This complex may be a heterodimer of progranulin and prosaposin.

In an embodiment of the instant invention, the cell lines of the invention further express a factor which stimulates secretion of progranulin or prosaposin from the cell line.

In a preferred embodiment, cell lines of the instant invention are contained within an implantable cell device that is then inserted into a patient in need of treatment. Examples of such a cell device can be found, generally, in U.S. Pat. Nos. 8,741,340; 9,121,037; 9,364,427; 9,669,154; 9,884,023; 10,835,664 and 10,888,526, all of which are hereby incorporated by reference. Such a device, when implanted inside of a patient, allows the alpha-synuclein antibody or antibody fragment, progranulin and prosaposin and GDNF secreted by the cell line to be efficiently delivered to the patient without the need for repeated traumas. As the factors are continuously produced, there is no need for formulation buffers and protein stability concerns. The stable cell line is also considered a single drug substance while secreting more than one effector molecule, which allows for new therapeutic interventions in difficult to treat diseases.

In a preferred embodiment, the implantable cell device comprises a semi-permeable membrane permitting the diffusion of molecules secreted from the cell line situated within said implantable cell device through said membrane. In a further embodiment, the semi-permeable membrane is immune-isolating to protect the cell line within from the patient's immune system. In another preferred embodiment, the implantable cell device comprises a matrix disposed within the semi-permeable membrane to promote efficient growth and survival of the cell line enclosed within.

In an embodiment, the implantable cell device may further comprise a means to implant the device inside of a patient in need of treatment. This implanting means may be a catheter. The device may be implanted into the patient in various tissue compartments and preferably intrathecally, intracerebroventricularly, or intracerebrally. Preferred targets for implantation include the striatum, the spinal canal and subarachnoid space of the patient.

In an embodiment, the implantable cell device may further comprise a vehicle to facilitate delivery of alpha-synuclein antibodies or antibody fragments, progranulin and prosaposin from the cell line to the desired location within the patient's body. In various embodiments, the vehicle is a pump or a syringe or associated catheter systems.

Cell lines of the invention may be useful in the treatment of neurological diseases or disorders, in particular, lysosomal storage disorder or neurodegenerative diseases that are disorders characterized by multiple pathologies. Neurological disorders treatable by cell lines of the invention include, but are not limited to, frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia (LBD), Parkinson's disease (PD), Multiple system atrophy (MSA) and lysosomal storage disorders. The lysosomal storage disorders that may be treated using the cell lines of the invention include, but are not limited to, Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease, Farber's disease, and combinations thereof.

Alpha-synuclein antibodies or antibody fragments and progranulin, prosaposin and complexes of progranulin and prosaposin produced by cell lines of the invention may also be purified for use as a therapeutic in the treatment of a neurological disorder. In this embodiment, the cell lines of the invention may be contained inside of a bioreactor to produce large quantities of alpha-synuclein antibodies or antibody fragments, progranulin, prosaposin and progranulin/prosaposin complexes. In a further embodiment, the progranulin and progranulin/prosaposin complexes are purified by several biochemical and chromatographic methods including, but is not restricted to, salt precipitation, protein A affinity chromatography, gel filtration and ion exchange chromatography. In yet a further embodiment, recombinant proteins isolated from cell lines of the invention can be administered as a therapeutic to a patient in need of treatment for a neurological disorder, as defined above. In various further embodiments, the therapeutic is administered by the use of a pump, syringe, or catheter system.

The inventors have further discovered that the complex of progranulin and prosaposin is present in body fluids, thus making it possible to monitor with inverted immune-based methods such as ELISA recognizing the complex. The absolute levels of extracellular progranulin/prosaposin complex levels may be a useful biomarker in the diagnosis and for the monitoring of drug exposure and treatment responses. In addition, the ratio of un-complexed progranulin or un-complexed prosaposin compared to progranulin/prosaposin complexes present in a fluid sample from a patient may provide important information and be a useful biomarker in the diagnosis of, but not restricted to, a neurological disorder or a means to assess the prognosis and progression of a neurological disorder, especially after treatment begins. The biomarkers of the instant application may also be used in the diagnosis and for the monitoring of an inflammatory disease, cancer and obesity-associated pathologies. Inflammatory diseases include, but are not limited to, cholelithiasis, fatty liver disease, endometriosis, inflammatory bowel disease, asthma, rheumatoid arthritis, chronic peptic ulcer, periodontitis, Crohn's disease, sinusitis, hepatitis, cardiovascular disease, arthritis, chronic obstructive pulmonary disease, encephalitis, meningitis, neuritis and pancreatitis. Obesity-associated pathologies include, but are not limited to, Type 2 diabetes mellitus, Type 1 diabetes, hyperlipidemia, insulin insensitivity, hyperglycemia, hyperinsulinemia, hypoinsulinemia, dyslipidemia, hypertension and atherosclerosis.

In various embodiments, the concentrations of un-complexed progranulin, un-complexed prosaposin, and progranulin/prosaposin complexes are determined by enzyme-linked immunosorbent assay (ELISA) or any other immune-based assay principles, such as electrochemiluminescence, e.g., the Meso Scale Discovery® technology (Meso Scale Diagnostics®, Rockville, Md.), Simoa® technology (Quanterix™ Corporation, Billerica, Mass.), HTRF® (homogenous time resolved fluorescence)(Cisbio Bioassays Societe Par Actions Simplifee a Associe Unique France Parc Marcel Boiteux B.P., Codolet, FR), Alphascreen® (PerkinElmer®, Waltham, Mass.) and/or a proximity ligation assay, however, alternative analytical methods may also be used. In various embodiments, the fluid sample may be plasma, cerebrospinal fluid, saliva, tear drops or urine.

### BRIEF DESCRIPTION OF THE DRAWINGS

FIG. 1 illustrates the results of PGRN/PSAP and PSAP/PGRN ELISA assays on conditioned media from different ECB cell lines. The assays are able to detect the presence of PGRN/PSAP complexes from ARPE-PGRN, ARPE-PGRN+PSAP co-transfected cell lines and from FLAG™-PGRN transfected cells (black arrows).

FIG. 2 are Western blots of PGRN and PSAP in crosslinked conditioned media from following ECB cell lines: ARPE-19 parental cell line (A), PGRN (56), PSAP (#7), and PGRN+PSAP+scFv81 co-expressing line (D5). Arrows indicate presence of PGRN/PSAP complexes that are consistent with the ELISA results depicted in FIG. 1.

FIG. 3 describes a sample of ELISAs on conditioned media from cultured, encapsulated cells overexpressing human progranulin, human prosaposin, or human prosaposin+progranulin (ARPE-19 cells/Sleeping beauty system). Progranulin overexpressing devices secrete mostly monomeric progranulin, but also progranulin/prosaposin complexes. Prosaposin overexpressing devices secrete only prosaposin. Prosaposin+progranulin over expressing ECB devices secrete mostly progranulin/prosaposin complexes. cl56 is a progranulin secreting device and D5 is a prosaposin+progranulin+scFv81 device.

FIGS. 4A-4C graphically depicts size exclusion chromatography of conditioned media from ARPE-19 cells overexpressing progranulin or progranulin+prosaposin+scFv81. Merge of raw data from FIG. 4A for cells overexpressing progranulin+prosaposin+scFv81 FIG. 4B and cells overexpressing progranulin FIG. 4C show that free progranulin is only detected in fractions from progranulin overexpressing cells whereas all secreted progranulin is in complex with prosaposin in conditioned media from cells overexpressing progranulin+prosaposin.

FIGS. 5 are direct ELISAs on conditioned media from cultured devices and cell lines secreting FLAG™-tagged scFV and Fc-scFv anti-alpha-synuclein targeting antibody fragments, respectively, and the immunocytochemistry results of alpha-synuclein targeting FLAG™-tagged scFv in cell lines selected for the overexpression of PGRN and PSAP and the alpha-synuclein targeting FLAG™-tagged scFv81 (top right) and cell lines overexpressing alpha-synuclein targeting peptides scFv81, scFv113, and scFv49. Illumination is FITC-anti FLAG™ and Alexa Fluor® 647-anti hIgG (Molecular Probes, Inc., Eugene, Oreg.) for cells overexpressing and secreting Fc-scFv-.

FIGS. 6A-6E illustrates the PGRN/PSAP assay (A) graphically depicts data generated from sandwich enzyme-linked immunosorbent assays (ELISA) detecting complexes of PGRN/PSAP derived from commercially available recombinant PGRN (Research and Diagnostic Systems, Minneapolis, Minn.) and PSAP (Abnova® GmbH, Taipei, TW) assembled in vitro (FIG. 6B and FIG. 6C) show that PGRN/PSAP complexes are present in human plasma (FIG. 6D) and in human CSF (FIG. 6E) and could be monitored with these assays. These assays detect complexes both by capturing for PGRN first FIG. 6B or PSAP first FIG. 6C, followed by detection antibodies specific for PSAP and PGRN, respectively.

FIG. 7 are images created by immunocytochemical analysis probing for PGRN (left), lysosomal protein GBA1 (top center), and PSAP (bottom center). Human patient primary GBA1-mutant fibroblasts (top row) and mouse primary cortical neurons (bottom row) were exposed to purified PGRN/PSAP complexes derived from conditioned media of cultured ARPE-PGRN+PSAP co-expressing cells. Merge (right column) shows efficient intracellular targeting of PGRN to the lysosomal protein GBA1 and purified PGRN+PSAP complexes to cortical neurons.

FIGS. 8A, 8B and 8C are graphical depictions of the activity of conditioned media and purified secreted factors from ARPE-PGRN, ARPE-PSAP, and ARPE-PGRN+PSAP cell lines. Primary mouse cortical neurons FIGS. 8A and 8C and human primary fibroblasts FIG. 8B were exposed to conditioned media from ARPE-19 cell lines FIG. 8A and purified PGRN and PGRN/PSAP complexes from the conditioned media from ARPE-19 cell lines FIGS. 8B and 8C and assayed for GBA1 activity using spectrometry.

FIGS. 9A and 9B are images of the safety of implantation of PGRN-devices by histopathology twenty four (24) weeks post-implantation. FIG. 9A shows broad distribution of PGRN around the implantation site (arrow). FIG. 9B shows no cell proliferation, inflammatory reactions, or infiltrating T-cells compared to control by probing for Ki67, GFAP, Iba1 and CD3.

FIGS. 10A-10D depict the in vivo activity of implantation of a cell device as measured by rescue of alpha-synuclein driven loss of tyrosine hydroxylase positive neurons in the striatum and behavioral improvement. FIGS. 10A are a selection of images of devices filled with PGRN, PSAP and PGRN-PSAP complex prevented loss of neurons as compared to non-device treated rats. FIG. 10B graphically illustrates the densitometric analysis of TH immunoreactivity presented as the difference between the ipsilateral and contralateral side. FIG. 10B shows examples of TH and alpha-synuclein IHC staining (alpha-synuclein overexpression was induced by unilateral injection in substantia nigra with AAV9 viruses carrying the human alpha-synuclein gene). FIG. 10C demonstrates how ECB-PGRN improves motor function in rats subjected to unilateral AAV9-alpha-synuclein gene injections in substantial nigra. FIG. 10D shows how ECB-PSAP and ECB-PGRN-PSAP complex improves motor function in rats subjected to unilateral AAV9-alpha-synuclein gene injection in substantia nigra.

FIGS. 11A and FIG. 11B depicts cell lines and in vivo activity of ARPE-cells co-expressing and secreting GDNF+PGRN or GDNF+PSAP or either factor alone. FIG. 11A shows GDNF and PSAP ELISA analysis of different cell lines expressing GDNF, PSAP and GDNF+PSAP, respectively. FIG. 11B graphically depicts the results of spontaneous forelimb placing use behavioral test for rats in the 6-OHDA model. ECB devices containing parental cells (placebo) or cells secreting either PSAP, PGRN, GDNF and PGRN or GDNF and PSAP were placed in the striatum. Rats were held with their limbs hanging unsupported and the length of their body parallel to the edge of a table. The rats were then raised to the side of a table so the whiskers of said rats made contact with the table. A naïve rat will typically respond by placing the forelimb on the top of the table. Asterisks indicate rats treated with placebo devices.

FIGS. 12A and 12B graphically represent the increased neurite outgrowth as visualized by immunocytochemistry probing for tubulin and increase in granulins, prosaposin, saposin C and GCase activity in human primary fibroblasts derived from control and GRN mutation carriers. Supplementation of cell media with either PGRN or PGRN/PSAP increases mean branch point count per neuron FIG. 12A and mean neurite length FIG. 12B. Supplementation of cell media of control and GRN-mutant derived fibroblasts with PGRN and PGRN/PSAP increase intracellular granulins FIG. 12C, Prosaposin FIG. 12D, Saposin C FIG. 12E and GCase activity FIG. 12F.

FIG. 13 is a selection of images of overlapping diffusion into the brain of PGRN and PSAP from rats injected with concentrated conditioned media from ARPE-PSAP cells mixed with PGRN-His. At higher magnification, intracellular co-localization of PGRN and PSAP were detected in these brains and also intracellular colocalization of His-like and PSAP-like immunoreactivity, suggesting that intracerebroventricular (ICV) administered PGRN/PSAP complexes are diffused into the brain and internalized by brain cells.

FIGS. 14A-14F illustrate distribution into the brain, internalization, and lysosomal targeting after ICV administration of conditioned media from ARPE-PSAP cells mixed with PGRN-His in mice and rats (FIG. 14A). ICV administration of purified PGRN, PGRN/PSAP complexes and PGRN-Fc in rats (FIGS. 14B-FIG. 14D). ICV implantation of devices secreting PGRN and PGRN/PSAP complexes in pigs followed by an ELISA analysis of brain lysates (FIG. E) and intrathecal administration of purified PGRN in pigs followed by ELISA analysis of brain lysates (FIG. F). Administered PGRN and PSAP were visualized by immunohistochemistry in mouse and rat (FIG. 14A-FIG. D) and by PGRN ELISA (FIG. 14E and FIG. 14F).

FIGS. 15A-FIG. 15C graphically represent the production of PGRN FIG. 15A, PSAP FIG. 15B, and PGRN+PSAP FIG. 15C after twelve (12) weeks implantation of encapsulated cell devices secreting the respective therapeutics in an AAV-alpha-synuclein rat Parkinson's disease model.

FIG. 16 is a graphic representation of a map of the pT2.CAn.PGRN plasmid used in construction of cell lines used in this application. Gene sequences are inserted between the left and right inverted repeat/direct repeat elements (IR/DR) and are integrated into the host genome by the Sleeping beauty transposase system. In this example, the genes to be inserted by the Sleeping beauty system are PGRN, Neo, and a promoter sequence (CA). The plasmid sequence is described as SEQ ID NO. 1.

### DETAILED DESCRIPTION OF THE INVENTION

There is a decrease in activity of the lysosomal enzyme glucocerebrosidase (GCase) encoded by the gene GBA1 in the brains of patients with frontotemporal dementia (FTD) and GRN-related frontotemporal dementia, Lewy body dementia associated with GBA1 mutations (LBD/GBA1), in Parkinson's Disease (PD) and in Gaucher's disease. Restoration of GCase activity is a major therapeutic goal for these indications. In addition, mutations in prosaposin have been linked to autosomal dominant inherited PD. Similarly, several damaging mutations in the progranulin encoding gene have been associated with PD. Extracellularly administered recombinant progranulin (PGRN), prosaposin (PSAP) and recombinant PGRN+PSAP complexes are internalized and colocalizes with GCase in lysosomes in human fibroblasts and increase GCase activity in primary cortical neurons (target cell type for FTD, LBD, advanced PD and ALS). In addition, conditioned media derived from ARPE-PGRN, ARPE-PSAP, and ARPE-PGRN+PSAP cells i.e., the therapeutic formulation of ECB-PGRN, ECB-PSAP and ECB-PGRN+PSAP therapies, increase GCase activity in primary cortical neurons. The data supports the use of either recombinant PGRN, PSAP or PGRN/PSAP or their corresponding ECB-therapies for stimulating and rescuing GCase activity in different human disease.

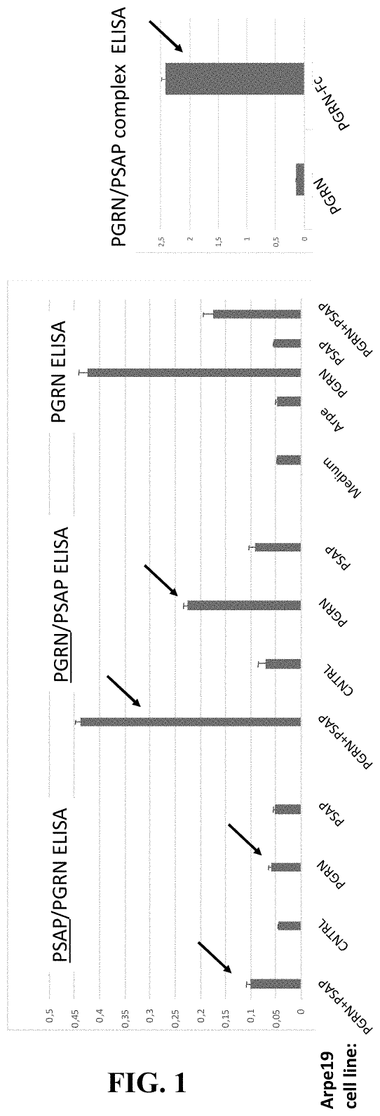

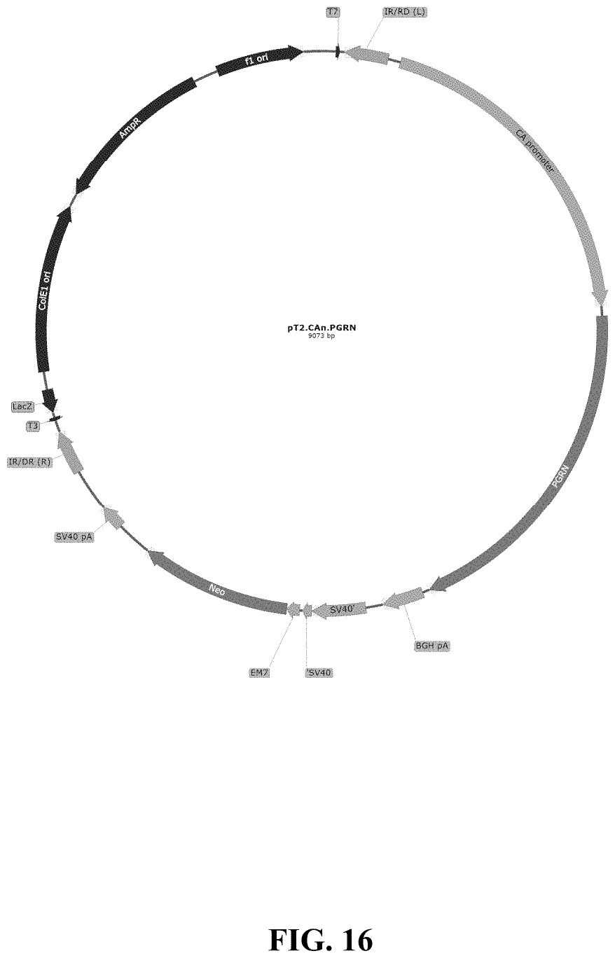

For the synucleinopathies LBD and PD, there is a strong link between decreased GCase activity and increased alpha-synuclein pathology/Lewy body pathology development. There is a strong rationale to combine GCase stimulation with an immunotherapy that targets alpha-synuclein misfolding in a single therapy. Importantly, the detrimental result of impaired GCase activity appears not restricted to Lewy body (LB) formation, but also impacts neuronal health in other ways leading to fatal consequences. This is suggested by the fact that disease progression, development of dementia and even lethality, besides accelerated Lewy body formation, is more aggressive and frequent in PD/GBA1 compared to PD without GBA1 mutations. Thus, multiple, therapeutic benefits, as a consequence of enhancing GCase activity combined with alpha-synuclein targeting immunotherapy, are expected. The novel alpha-synuclein targeting antibody fragments of the instant application were designed to interfere with alpha-synuclein pathology development at several levels: inhibit aggregation, bind to multiple alpha-synuclein species (monomers, oligomers, fibrils) and to exhibit a broad epitope coverage, in order to be as efficient as possible in blocking the development of alpha-synuclein pathology and to induce a sink effect. (See Methods point 7 that demonstrates the feasibility, proof, of generation of a clonal ARPE-19 cell line secreting PGRN, PSAP and an anti-alpha-synuclein targeting antibody fragment.) This cell line evidences the therapeutic effects in 8-12 week studies in two rat animal models of PD (Parkinson alpha-synuclein and 6-OHDA models). To this end, the PGRN, PSAP and PGRN+PSAP+anti-alpha-synuclein therapies have a positive impact on behavior in both models. As discussed, the GCase stimulatory factors progranulin and prosaposin and the anti-alpha-synuclein immunotherapy, mediate neuroprotection at different levels of the PD and LBD pathological cascades. In addition to these activities, there is a need to restore function of already damaged neurons. A therapeutic mediating neurorestorative activity would complement the aforementioned neuroprotective therapeutics in order to achieve an as clinically meaningful therapy as possible. GDNF is a secreted factor that has been demonstrated to mediate neurorestorative activity in animal models and in some patients. One embodiment of the claimed invention is directed to a unique therapy composed of multiple neuroprotective activities combined with neurorestorative activity, all in a single therapy. As demonstrated by the data set forth in FIGS. 1-16, behavioral improvement in the 6-OHDA rat model of Parkinson's disease after 2-12 weeks of treatment was observed with the following ECB therapies: ARPE-PGRN, ARPE-PGRN+PSAP+anti-alpha-synuclein, ARPE-PGRN+GDNF and ARPE-PSAP+GDNF. Thus, the data evidences the ability to generate efficacious ECB-therapies based on ARPE-cell lines co-expressing several therapeutic factors targeting both lysosomal signaling and neurorestoration. Also, therapeutic activity (behavioral improvement) of the ECB-PGRN, ECB-PSAP and PGRN+PSAP+anti-alpha-synuclein combinations, in a rat model of Parkinson's disease/synucleinopathies, a human alpha-synuclein overexpression model, was also demonstrated providing preclinical proof of concept for these five different therapies for Parkinson's disease and other synucleinopathies, is supported by the data set forth in FIGS. 1-16.

GRN-related frontotemporal dementia (FTD) is caused by haploinsufficiency in the secreted factor progranulin, which mediates signaling both extracellularly via different cell surface receptors and intracellularly, at the level of the lysosome where it regulates multiple factors such as Cathepsin D, PSAP and GCase. To restore PGRN signaling is a primary therapeutic goal in GRN-related FTD. PGRN deficiency in FTD/GRN is accompanied by decreased neuronal PSAP levels as well as reduced GCase activity. To what extent the levels of intracellular PGRN/PSAP complexes are affected in FTD/GRN, or to what extend the disease impacts the extracellular levels of free PSAP and the PGRN/PSAP complex are not known. FIGS. 1-16 demonstrate that PGRN is in complex with PSAP in human plasma and CSF and therefore a biomarker of strong relevance for diagnosis of FTD/GRN and other disorders implicating PGRN, PSAP and GCase signaling. It is conceivable that both the absolute levels of circulating PGRN/PSAP and the relative ratio of circulating PGRN/PSAP versus free circulating PGRN and free circulating PSAP are important for the diagnosis, prognosis and for monitoring drug exposure and treatment responses. The discovery that PGRN/PSAP is present in CSF and in direct contact with the human cortex is important and demonstrates that both PGRN and the PGRN/PSAP complex are endogenous extracellularly expressed molecules in the primarily affected brain area subjected to neurodegeneration due to PGRN haploinsufficiency in FTD/GRN pathogenesis. Both PGRN and the PGRN/PSAP complex diffuses into the brain after IT or ICV administered recombinant proteins or ECB based delivery of PGRN and PGRN/PSAP complexes. Furthermore, FIGS. 1-16 show that the intracerebroventricularly-administered PGRN/PSAP mixtures are colocalized with neurites in the brain. Collectively, the data set forth in FIGS. 1-16 conclusively shows that IT and ICV administered PGRN, PSAP and PGRN/PSAP complexes efficiently diffuse into the brain and target neurons from the CSF compartment, rescuing intracellular granulin, prosaposin and saposin C levels, as well as GCase activity. This data supports the need for specific assays specific to PGRN, PSAP and the PGRN/PSAP complex as accompanying biomarkers for therapies aimed to stimulate and/or restore PGRN and PSAP levels and signaling in disease.

PGRN/PSAP complexes mediate neurotrophic activity: FIGS. 1-16 evidence the stimulation of neurite outgrowth of rodent primary cortical neurons. Both PGRN and PSAP have been shown to be neurotrophic factors. The data set forth in FIGS. 1-16 suggests and supports the use of PGRN/PSAP as a neuroprotective therapeutic in FTD/GRN, FTD/TDP, i.e., FTD with TDP pathology in general (not necessary GRN haploinsufficiency), LBD/GBA1, PD and PD/GBA1 and ALS.

Neuronal ceroid lipofuscinosis (NCL) is a lysosomal storage disorder caused by deficiency in PGRN (100% deficiency). Extracellularly administered PGRN or PGRN/PSAP complexes are internalized and localized to lysosomes. In addition, conditioned media from ARPE-PGRN and ARPE-PGRN+PSAP brings the same activity, thus, ECB-based administration of PGRN or PGRN/PSAP is used to rescue lysosomal PGRN signaling in NCL.

In ALS, FTD and AD, TDP pathology is the most common proteinopathy. PGRN deficiency in FTD/GRN results in TDP pathology associated neurodegeneration. Crossing a mouse model of ALS/TDP with mouse overexpressing PGRN resulted in a less severe ALS-like phenotype, i.e., PGRN appears to be therapeutic for TDP associated ALS. Based on this prior art, PGRN/PSAP maybe used as a therapeutic for ALS/TDP, as well as for FTD/TDP and AD. In particular, IT administration of PGRN or PGRN/PSAP, targeting the spinal canal and cortex, key regions in ALS pathogenesis (and FTD, LBD and AD), holds great promise as an effective treatment. The data set forth in FIGS. 1-16 shows that IT-administered PGRN and ICV-administered PGRN and PGRN/PSAP complexes diffuse into the brain and targets areas of key relevance for ALS, FTD, LBD and AD. No one has ever demonstrated CSF to brain diffusion of either PGRN, PSAP or PGRN/PSAP.

### EXEMPLIFICATION

### Example 1: ARPE-PGRN Cell Line Generation

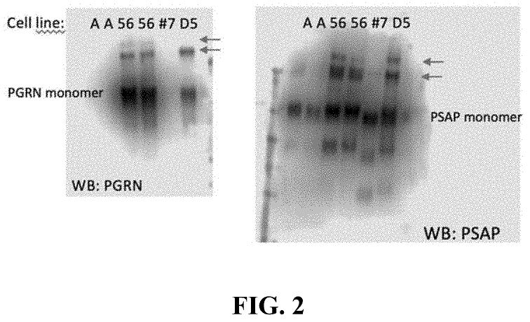

ARPE-19 cells (ATCC®, Manassas, Va.) were grown to 70% confluency in F12/DMEM media (Dulbecco's Modified Eagle Medium: Nutrient Mixture F-12, Thermo Fisher Scientific®, Waltham, Mass.) (Gibco®, cat no. 31331-028, supplemented with 10% FCS and penicillin/streptomycin (PEST)) (hereinafter referred to as “complete media”). On the day of transfection, the cell media was replaced with serum free media and the cells were transfected with a plasmid constitutively expressing the consensual human progranulin gene under a chicken beta-actin promoter and CMV enhancer. The recombinant expression construct also contained the neomycin selection gene and was flanked by sleeping beauty transposable elements. Transient expression of a sleeping beauty transposase was used to stably integrate copies of the PGRN cDNA transgene construct (Sleeping beauty transposon system). The plasmids were introduced using Promega® Fugene 6® transfection kit (Promega® Corporation, Madison, Wis.), according to the manufacturer's instructions. Forty-eight (48) hours post transfection, the cells were split 1:10 and seeded in 10 cm² tissue culture dishes in the presence of complete media supplemented with 800 μg/ml Geneticin® (Thermo Fisher Scientific®, Waltham, Mass.) to select clones expressing the neomycin selection marker. Fourteen (14) days later, individual colonies were harvested and expanded in complete media supplemented with 800 μg/ml Geneticin® (Thermo Fisher Scientific®, Waltham, Mass.). The different clonal cell lines were analyzed for secreted PGRN using the R&D Systems® anti-human PGRN DuoSet® kit (R&D Systems®, Minneapolis, Minn.). Moreover, ARPE-PGRN cells were also analyzed for secretion of PGRN/PSAP complexes (See sections 12 and 13 for methods of PGRN/PSAP complex monitoring). Encapsulated ARPE-PGRN cells secrete both PGRN and PGRN/PSAP complexes as determined with the aforementioned PGRN and PGRN/PSAP complex assays (FIG. 1) and by crosslinking of conditioned media followed by western blot analysis using PGRN- and PSAP-directed antibodies (FIG. 2). ARPE-PGRN clone #56 was chosen for further characterization in vitro and encapsulated for in vitro and in vivo therapy evaluation.

### Example 2: ARPE-PSAP Cell Line Generation

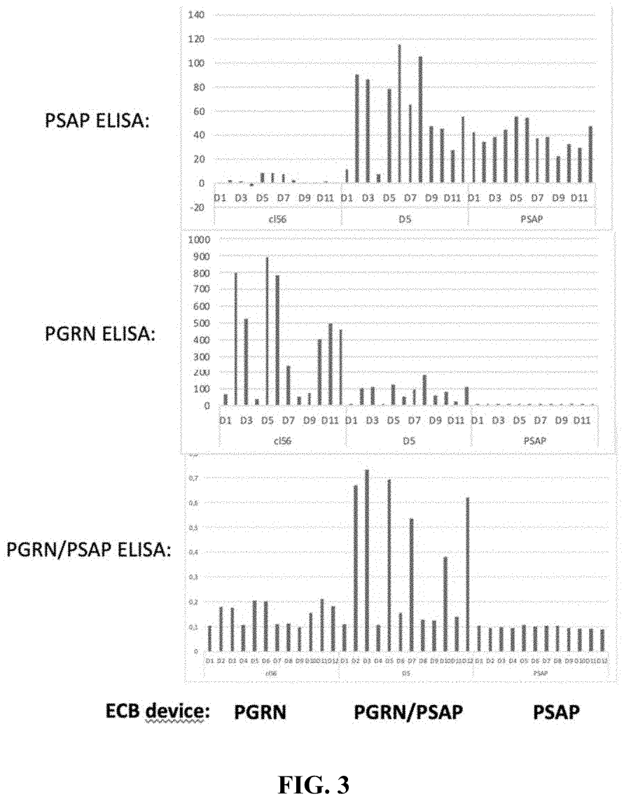

ARPE-19 cells (ATCC®, Manassas, Va.) were grown to 70% confluency in complete media. On the day of transfection, the cell media was discarded and replaced with serum free media. The cells were transfected with a plasmid (Sleeping beauty transposon system) encoding human PSAP cDNA using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 800 μg/ml Geneticin® (Thermo Fisher Scientific®, Waltham, Mass.). Fourteen (14) days later, individual colonies were harvested and expanded in complete media supplemented with 800 μg/ml Geneticin® (Thermo Fisher Scientific®, Waltham, Mass.). The different clonal ARPE-PSAP cell lines were analyzed for secreted PSAP using an ELISA assay (Thermo Fisher Scientific®, Waltham, Mass.), described in section 11. Encapsulated ARPE-PSAP cells secrete PSAP whereas neither PGRN nor PGRN/PSAP complexes could be detected FIG. 3.

### Example 3: ARPE-PGRN+PSAP Cell Line Generation

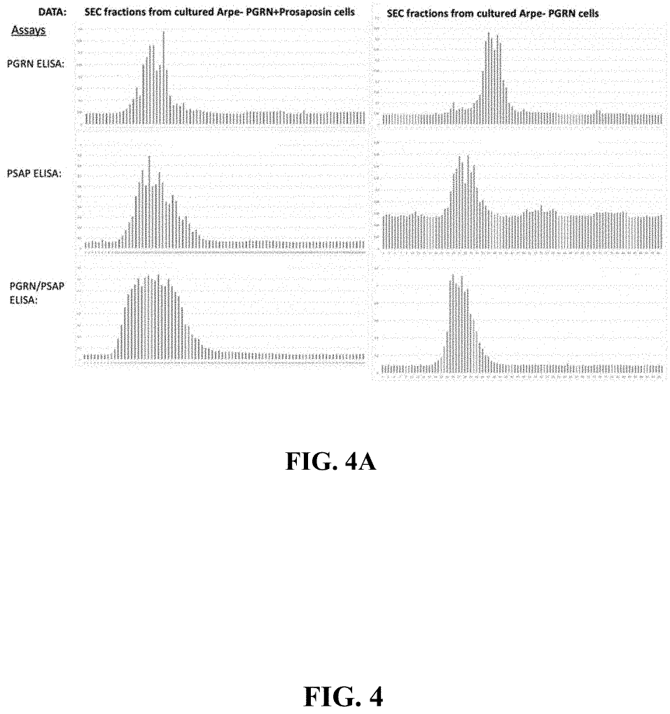

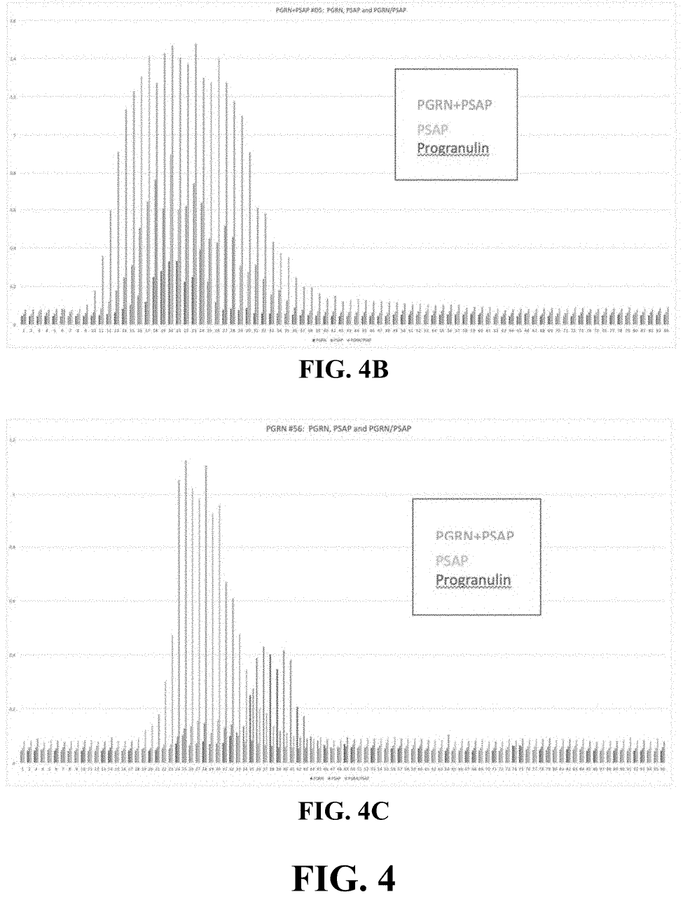

ARPE-19-PGRN #56 cells were grown to 70% confluency in complete media and then transfected with a PSAP-encoding plasmid, as described in section 2, with the exception that the PSAP encoding plasmid had the G418 selection gene replaced with a hygromycin selection gene. Generation of clonal cell lines were accomplished as above, besides that both Geneticin® (Thermo Fisher Scientific®, Waltham, Mass.) (800 μg/ml) and Hygromycin™ (Sigma-Aldrich®, St. Louis, Mo.) (500 μg/ml) were included in the media. Clonal cell lines expressing both PGRN and PSAP were identified with ELISA (Thermo Fisher Scientific®, Waltham, Mass.). Encapsulated ARPE-PGRN+PSAP cells secrete mostly PGRN/PSAP complexes as assessed by analysis by of different fractions derived from size exclusion chromatography of conditioned media from ARPE-PGRN+PSAP cells FIG. 4.

### Example 4: Alpha-Synuclein Targeting ARPE-scFv81 Cell Line Generation

ARPE-19 cells (ATCC®, Manassas, Va.) were grown to 70% confluency in complete media. On the day of transfection, the cell media was discarded and replaced with serum free media. The cells were transfected with a plasmid (Sleeping beauty transposon system) encoding the construct (signalpeptide-scFv81-Flag-His)3):

(SEQ ID NO: 15)
MGILPSPGMPALLSLVSLLSVLLMGCVALPEVQLLESGGGLVQPGGSLR
LSCAASGFTFSSYAMSWVRQAPGKGLEWVSSIYGSGGYTSYADSVKGRF
TISRDNSKNTLYLQMNSLRAEDTAVYYCARTYGGRFDYWGQGTLVTVSS
GGGGSGGGGSGGGGSDIQMTQSPSSLSASVGDRVTITCRASQSISSYLN
WYQQKPGKAPKLLIYAASSLQSGVPSRFSGSGSGTDFTLTISSLQPEDF
ATYYCQQYTYPPTFGQGTKLEIKRTDYKDHDGDYKDHDIDYKDDDDKAA
AHHHHHH

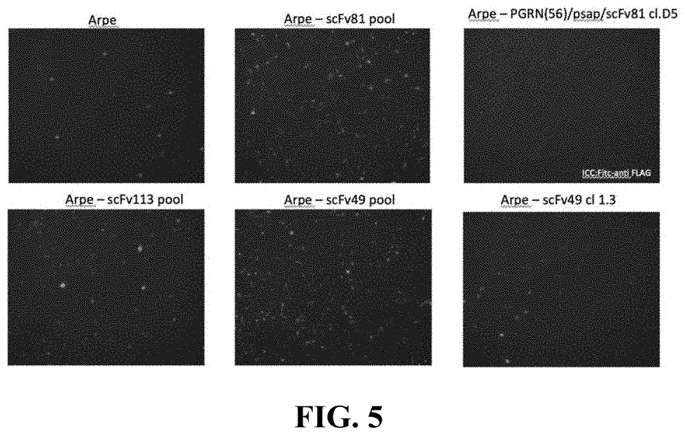

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 800 μg/ml Geneticin® (Thermo Fisher Scientific®, Waltham, Mass.). Fourteen (14) days later, individual colonies were harvested and expanded in complete media supplemented with 800 μg/ml Geneticin® (Thermo Fisher Scientific®, Waltham, Mass.) (FIG. 5).

### Example 5: Alpha-Synuclein Targeting ARPE-scFv49 Cell Line Generation

ARPE-19 cells (ATCC®, Manassas, Va.) were grown to 70% confluency in complete media. At day of transfection, the cell media was discarded and replaced with serum free media. The cells were transfected with a plasmid (Sleeping beauty transposon system) encoding the construct (signalpeptide-scFv49-Flag-His)4):

(SEQ ID NO: 13)
MGILPSPGMPALLSLVSLLSVLLMGCVALPEVQLLESGGGLVQPGGSLR
LSCAASGFTFSSYAMSWVRQAPGKGLEWVSAISGSGGSTYYADSVKGRF
TISRDNSKNTLYLQMNSLRAEDTAVYYCARSYSAFDYWGQGTLVTVSSG
GGGSGGGGSGGGGSDIQMTQSPSSLSASVGDRVTITCRASQSISSYLNW
YQQKPGKAPKLLIYAASSLQSGVPSRFSGSGSGTDFTLTISSLQPEDFA
TYYCQQITGYLFTFGQGTKLEIKRTDYKDHDGDYKDHDIDYKDDDDKAA
AHHHHHH

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 800 μg/ml Geneticin® (Thermo Fisher Scientific®, Waltham, Mass.). Fourteen (14) days later, individual colonies were harvested and expanded in complete media supplemented with 800 μg/ml Geneticin® (Thermo Fisher Scientific®, Waltham, Mass.). Expression and secretion of scFv49 was observed (FIG. 5).

### Example 6: Alpha-Synuclein Targeting ARPE-scFv113 Cell Line Generation

ARPE-19 cells (ATCC®, Manassas, Va.) were grown to 70% confluency in complete media. On the day of transfection, the cell media was discarded and replaced with serum-free media. The cells were transfected with a plasmid (Sleeping beauty transposon system) encoding the construct (signalpeptide-scFv113-Flag-His)5):

(SEQ ID NO: 17)
MGILPSPGMPALLSLVSLLSVLLMGCVALPEVQLLESGGGLVQPGGSLR
LSCAASGFTFYGSGMSWVRQAPGKGLEWVSGISSYGGSTYYADSVKGRF
TISRDNSKNTLYLQMNSLRAEDTAVYYCARANYWHSSLDYWGQGTLVTV
SSGGGGSGGGGSGGGGSDIQMTQSPSSLSASVGDRVTITCRASQSISSY
LNWYQQKPGKAPKLLIYAASSLQSGVPSRFSGSGSGTDFTLTISSLQPE
DFATYYCQQSAGLLTFGQGTKLEIKRTDYKDHDGDYKDHDIDYKDDDDK
AAAHHHHHH

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 800 μg/ml Geneticin® (Thermo Fisher Scientific®, Waltham, Mass.). Fourteen (14) days later, individual colonies were harvested and expanded in complete media supplemented with 800 μg/ml Geneticin® (Thermo Fisher Scientific®, Waltham, Mass.) (FIG. 5).

### Example 7: ARPE-PGRN+PSAP+scFv81 Cell Line Generation

Clonal ARPE-PGRN cells were grown to 70% confluency in complete media and then transfected with a PSAP encoding plasmid, as described in section 3, and a plasmid (Sleeping beauty transposon system) encoding the construct (signalpeptide-scFv81-Flag-His) described in section 5. Generation of clonal cell lines were accomplished by culturing the transfectants in presence of Geneticin® (Thermo Fisher Scientific®, Waltham, Mass.) (800 μg/ml) and Hygromycin™ (Sigma-Aldrich®, St. Louis, Mo.) (500 μg/ml). Clonal cell lines expressing PGRN, PSAP and scFv81 were identified with immunocytochemical analysis (ICC). Clone D5 (#D5), expressing PGRN, PSAP and scFv81 was selected for further analysis (FIG. 5).

### Example 8: Alpha-Synuclein Targeting ARPE-Fc-scFv81 Cell Line Generation

ARPE-19 cells (ATCC®, Manassas, Va.) were grown to 70% confluency in complete media. On the day of transfection, the cell media was discarded and replaced with serum free media. The cells were transfected with the plasmid described in section 3 (Sleeping beauty transposon system, Hygromycin™ (Sigma-Aldrich®, St. Louis, Mo.) selection gene) with a cDNA encoding the following peptide (signalpeptide-Fc-scFv81)6):

(SEQ ID NO: 7)
MPLLLLLPLLWAGALAEVQLLESGGGLVQPGGSLRLSCAASGFTFSSYA
MSWVRQAPGKGLEWVSSIYGSGGYTSYADSVKGRFTISRDNSKNTLYLQ
MNSLRAEDTAVYYCARTYGGRFDYWGQGTLVTVSSGGGGSGGGGSGGGG
SDIQMTQSPSSLSASVGDRVTITCRASQSISSYLNWYQQKPGKAPKLLI
YAASSLQSGVPSRFSGSGSGTDFTLTISSLQPEDFATYYCQQYTYPPTF
GQGTKLEIKGGGGSKPCICTGSEVSSVFIFPPKPKDVLTITLTPKVTCV
VVDISQDDPEVHFSWFVDDVEVHTAQTRPPEEQFNSTFRSVSELPILHQ
DWLNGRTFRCKVTSAAFPSPIEKTISKPEGRTQVPHVYTMSPTKEEMTQ
NEVSITCMVKGFYPPDIYVEWQMNGQPQENYKNTPPTMDTDGSYFLYSK
LNVKKEKWQQGNTFTCSVLHEGLHNHHTEKSLSHSPGK

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 500 μg/ml Hygromycin™ (Sigma-Aldrich®, St. Louis, Mo.). Fourteen (14) days later, individual colonies were harvested and expanded in complete media supplemented with 500 μg/ml Hygromycin™ (Sigma-Aldrich®, St. Louis, Mo.).

### Example 9: Alpha-Synuclein Targeting ARPE-Fc-scFv49 Cell Line Generation

ARPE-19 cells (ATCC®, Manassas, Va.) were grown to 70% confluency in complete media. On the day of transfection, the cell media was discarded and replaced with serum free media. The cells were transfected with the plasmid described in section 3 (Sleeping beauty transposon system, Hygromycin™ (Sigma-Aldrich®, St. Louis, Mo.) selection gene) with a cDNA encoding the following peptide (signalpeptide-Fc-scFv49)7):

(SEQ ID NO: 11)
MPLLLLLPLLWAGALAEVQLLESGGGLVQPGGSLRLSCAASGFTFSSYA
MSWVRQAPGKGLEWVSAISGSGGSTYYADSVKGRFTISRDNSKNTLYLQ
MNSLRAEDTAVYYCARSYSAFDYWGQGTLVTVSSGGGGSGGGGSGGGGS
DIQMTQSPSSLSASVGDRVTITCRASQSISSYLNWYQQKPGKAPKLLIY
AASSLQSGVPSRFSGSGSGTDFTLTISSLQPEDFATYYCQQITGYLFTF
GQGTKLEIKGGGGSKPCICTGSEVSSVFIFPPKPKDVLTITLTPKVTCV
VVDISQDDPEVHFSWFVDDVEVHTAQTRPPEEQFNSTFRSVSELPILHQ
DWLNGRTFRCKVTSAAFPSPIEKTISKPEGRTQVPHVYTMSPTKEEMTQ
NEVSITCMVKGFYPPDIYVEWQMNGQPQENYKNTPPTMDTDGSYFLYSK
LNVKKEKWQQGNTFTCSVLHEGLHNHHTEKSLSHSPGK

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 500 μg/ml Hygromycin™ (Sigma-Aldrich®, St. Louis, Mo.). Fourteen (14) days later, individual colonies were harvested and expanded in complete media supplemented with 500 μg/ml Hygromycin™ (Sigma-Aldrich®, St. Louis, Mo.) Expression and secretion of Fc-scFv49 was observed (FIG. 5).

### Example 10: Alpha-Synuclein Targeting ARPE-Fc-scFv113 Cell Line Generation

ARPE-19 cells (ATCC®, Manassas, Va.) were grown to 70% confluency in complete media. On the day of transfection, the cell media was discarded and replaced with serum free media. The cells were transfected with the plasmid described in section 3 (Sleeping beauty transposon system, Hygromycin™ (Sigma-Aldrich®, St. Louis, Mo.) selection gene) with a cDNA encoding the following peptide (signalpeptide-Fc-scFv113)8):

(SEQ ID NO: 9)
MPLLLLLPLLWAGALAEVQLLESGGGLVQPGGSLRLSCAASGFTFYGSG
MSWVRQAPGKGLEWVSGISSYGGSTYYADSVKGRFTISRDNSKNTLYLQ
MNSLRAEDTAVYYCARANYWHSSLDYWGQGTLVTVSSGGGGSGGGGSGG
GGSDIQMTQSPSSLSASVGDRVTITCRASQSISSYLNWYQQKPGKAPKL
LIYAASSLQSGVPSRFSGSGSGTDFTLTISSLQPEDFATYYCQQSAGLL
TFGQGTKLEIKGGGGSKPCICTGSEVSSVFIFPPKPKDVLTITLTPKVT
CVVVDISQDDPEVHFSWFVDDVEVHTAQTRPPEEQFNSTFRSVSELPIL
HQDWLNGRTFRCKVTSAAFPSPIEKTISKPEGRTQVPHVYTMSPTKEEM
TQNEVSITCMVKGFYPPDIYVEWQMNGQPQENYKNTPPTMDTDGSYFLY
SKLNVKKEKWQQGNTFTCSVLHEGLHNHHTEKSLSHSPGK

using the Promega® Fugene 6® transfection kit (Promega® Corporation, Madison, Wis.), according to the manufacturer's instructions. Forty-eight (48) hours post transfection, the cells were split 1:10 and plated in 10 cm² tissue culture dishes in the presence of complete media supplemented with 500 μg/ml Hygromycin™ (Sigma-Aldrich®, St. Louis, Mo.). Fourteen (14) days later, individual colonies were harvested and expanded in complete media supplemented with 500 μg/ml Hygromycin™ (Sigma-Aldrich®, St. Louis, Mo.).

### Example 11: ARPE-GDNF+PSAP Cell Line Generation

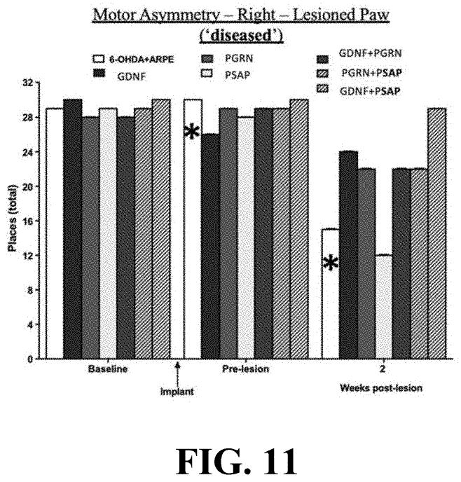

ARPE-19-GDNF cells were grown to 70% confluency in complete media and then transfected with a PSAP-encoding plasmid, as described in section 2, with the exception that the PSAP-encoding plasmid has the G4 1 8 selection gene replaced with a hygromycin selection gene. Generation of clonal cell lines was accomplished as discussed previously, besides that both Geneticin® (Thermo Fisher Scientific®, Waltham, Mass. )(800 μg/ml) and Hygromycin™ (Sigma-Aldrich®, St. Louis, Mo.)(500 μg/ml) were included in the media. Clonal cell lines expressing both GDNF and PSAP were identified with ELISA (Thermo Fisher Scientific®, Waltham, Mass.) as illustrated in FIG. 11.

### Example 12: Human PSAP ELISA Assay

The capture antibody (mouse mAb Abnova® cat no. H00005660-M01, 0.43 mg/ml (Abnova® GmbH, Taipei, TW)) was diluted 1:500 in phosphate buffered saline (PBS) (HyClone® Laboratories, Inc., South Logan, Utah) and 50 μI/well were added to Nunc MaxiSorp™ plates (cat no. 442404 (Thermo Fisher Scientific®, Waltham, Mass.)). The plates were incubated at room temp (RT) overnight (ON). The reaction mixture was then discarded and the wells were subsequently washed three (3) times in PBS/Tween20 (0.1%) (Sigma-Aldrich®, St. Louis, Mo.) prior to the addition of 150 μl blocking solution (PBS/Tween/BSA (2%)) (Alfa Aesar®, Tewksbury, Mass.) to each well. After a two (2) hour incubation at RT, the plates were washed twice in PBS/Tween20 (0.1%) (Sigma-Aldrich®, St. Louis, Mo.) prior to the addition of samples that were diluted in PBS/Tween20 (0.1%)/1% Bis(trimethylsilyl)acetamide (BSA) (Me₃SiNC(OSiMe₃)Me) (Alfa Aesar®, Tewksbury, Mass.). The binding reactions were left for two (2) hours at RT and then removed, followed by three (3) washes in PBS/Tween20 (0.1%) (Sigma-Aldrich®, St. Louis, Mo.). Then, 50 μl/well of detection antibody (Rb anti-PSAP, HPA004426, 0.1 mg/ml, diluted 1:300 in PBS/Tween20 (0.1%)/BSA (1%) (Alfa Aesar®, Tewksbury, Mass.)) were added and the reactions left for two (2) hours at RT. After another three (3) washes, 50 μl/well of horseradish peroxidase (HRP)-conjugated anti-Rb IgG antibody were added and the plates were incubated for one (1) hour at RT. Finally, the plates were washed three (3) times in PBS/Tween20 (0.1%) (Sigma-Aldrich®, St. Louis, Mo.) and HRP activity monitored using 1 Step™ TMB Ultra reagent (Thermo Fisher Scientific®, Waltham, Mass.) followed by addition of one (1) volume 2M H₂SO₄ to stop the reactions. Absorbance at 450 nm was subsequently monitored using a Molecular Devices® microplate reader (Promega® Corporation, Madison, Wis.). Human recombinant PSAP was used as standard (ABCAM®, cat nr: 203534 (ABCAM® PLC Co., Cambridge, UK)).

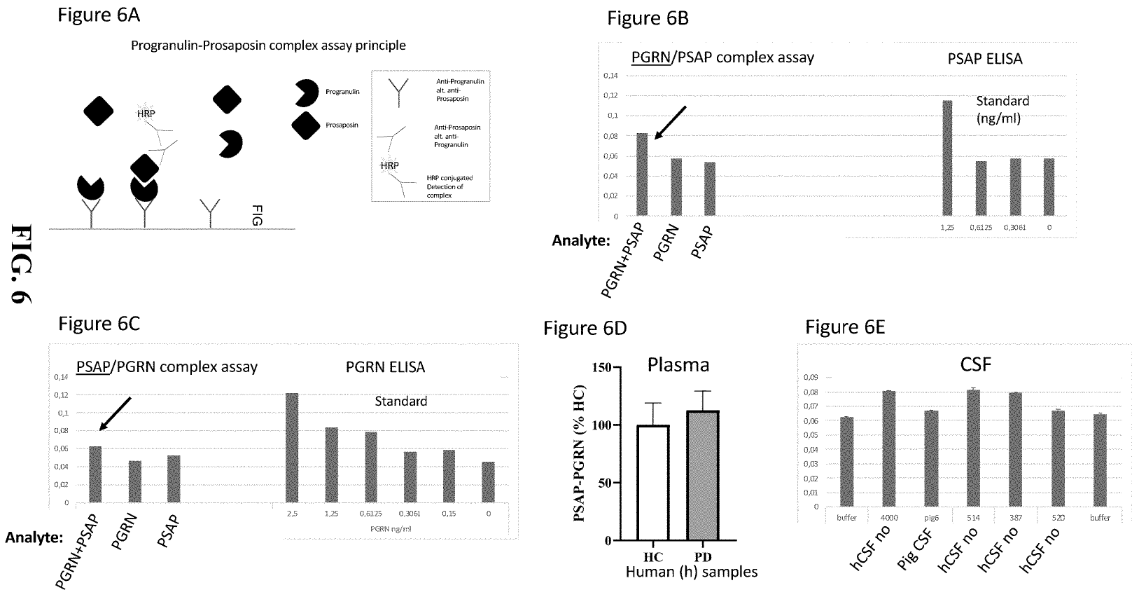

### Example 13: Human PGRN/PSAP Complex Assay (as Illustrated in FIG. 6A)

The capture antibody, anti-PGRN antibody (hPGRN ELISA DuoSet® kit (R&D Systems®, Minneapolis, Minn.)) was diluted according to the manufacturer's instructions and 50 μl/well were added in Nunc MaxiSorp™ 96-well plates (cat no. 442404 (Thermo Fisher Scientific®, Waltham, Mass.)). The reactions were incubated on at room temperature (RT). The reactions were then discarded and the plates washed three (3) times in PBS/Tween20 (0.1%) (Sigma-Aldrich®, St. Louis, Mo.). Approximately, 150 μl blocking solution (PBS/Tween/BSA (2%) (Alfa Aesar®, Tewksbury, Mass.)) were subsequently added and the plates incubated for two (2) hours at RT. The plate was then washed twice in PBS/Tween20 (0.1%) (Sigma-Aldrich®, St. Louis, Mo.) prior to addition of samples that were diluted in PBS/Tween20 (0.1%)/BSA (1%) (Alfa Aesar®, Tewksbury, Mass.). After two (2) hours of incubation at RT the plates were washed three (3) times prior to addition of an antibody recognizing PSAP (anti-PSAP, HPA004426, 0.1 mg/ml, diluted 1:300 in PBS/Tween20 (0.1%)/BSA (1%) (Alfa Aesar®, Tewksbury, Mass.)). Reactions were incubated for two (2) hours at RT. After three (3) washes, a horseradish peroxidase (HRP)-conjugated anti-Rb IgG antibody was added and the reactions incubated for one (1) hour at RT. Finally, the plates were washed three (3) times and HRP activity was monitored using 1 Step™ TMB Ultra reagent (Thermo Fisher Scientific®, Waltham, Mass.) followed by addition of 1 volume 2M H₂SO₄ to stop the reactions. Absorbance was finally read at 450 nm. As standard, purified PGRN/PSAP complexes derived from conditioned media from ARPE-PGRN/PSAP cells were used. PGRN/PSAP was diluted in PBS/Tween20 (0.1%)/BSA (1%) (Alfa Aesar®, Tewksbury, Mass.). As evidenced by FIG. 6, monitoring of PGRN/PSAP complexes derived from recombinant PGRN and PSAP assembled in vitro. PGRN/PSAP complexes, purified from conditioned cell culture media, secreted from ARPE-PGRN and ARPE-PGRN+PSAP cells and devices, are detectable in human plasma and CSF (FIG. 6) by this PGRN/PSAP complex ELISA (all CSF samples were diluted 1:1 in PBS/Tween20 (0.1%)/BSA (1%) (Alfa Aesar®, Tewksbury, Mass.)).

### Example 14: Human PSAP/PGRN Complex Assay (as Illustrated in FIG. 6A)

The capture antibody, mouse mAb anti-PSAP antibody (Abnova® cat no. H00005660-M01, 0.43 mg/ml (Abnova® GmbH, Taipei, TW)), was diluted 1:500 in PBS (HyClone® Laboratories, Inc., South Logan, Utah) and 50 μl/well Nunc MaxiSorp™ 96-well plates (cat no. 442404 (Thermo Fisher Scientific®, Waltham, Mass.)) were added. After incubating the plates at room temperature (RT), the reaction mixture was discarded and the plates were washed three (3) times in PBS/Tween20 (0.1%) (Sigma-Aldrich®, St. Louis, Mo.). Then, 150 μl blocking solution (PBS/Tween/BSA (2%) (Alfa Aesar®, Tewksbury, Mass.)) was added and the plates incubated for two (2) hours at room temp. The plates were then washed twice in PBS/Tween20 (0.1%) (Sigma-Aldrich®, St. Louis, Mo.) prior to addition of samples, which were diluted in PBS/Tween20 (0.1%)/BSA (1%) (Alfa Aesar®, Tewksbury, Mass.). The reactions were left for two (2) hours at RT and the removed, followed by three (3) washes in PBS/Tween20 (0.1%) (Sigma-Aldrich®, St. Louis, Mo.). Then, 50 μl/well of detection antibody (biotinylated anti-PGRN antibody, R&D Systems® hPGRN ELISA DuoSet® kit (R&D Systems®, Minneapolis, Minn.), diluted according to the manufacturer's instructions in PBS/Tween20 (0.1%)/BSA (1%)(Alfa Aesar®, Tewksbury, Mass.)) were added and the reactions left for two (2) hours at RT. After another three (3) washes, 50 μl/well of HRP-conjugated Streptavidin (Thermo Fisher Scientific®, Waltham, Mass.) were added and the plates incubated for thirty (30) minutes at RT. Finally, the plates were washed 3 times and HRP activity monitored using 1 Step™ TMB Ultra reagent (Thermo Fisher Scientific®, Waltham, Mass.) followed by the addition of one (1) volume of 2M H₂SO₄ to stop the reactions. Absorbance was finally read at 450 nm. FIG. 6 shows monitoring of PSAP/PGRN complexes derived from recombinant PGRN and PSAP assembled in vitro. PSAP/PGRN complexes, secreted from ARPE-PGRN and ARPE-PGRN+PSAP cells are also detectable by this PSAP/PGRN complex ELISA (FIG. 1). The release of progranulin/prosaposin complexes from ARPE, ARPE-PGRN, ARPE-PSAP and ARPE-PGRN+PSAP+scFv81 cells was further analyzed by a combined crosslinking/western blot experiment using conditioned media from the aforementioned cell lines as analyte (FIG. 2). Confluent cells cultures were conditioned for ninety-six (96) hours in FreeStyle™ 293 Expression Medium (Invitrogen/Thermo Fisher Scientific®, Carlsbad, Calif.). BS3 crosslinker (25 mM stock in H₂O) (Pierce Biotechnology/Thermo Fisher Scientific®, Waltham, Mass.) was then added to a final concentration of 1 mM and the reactions were kept at room temperature (RT) for one (1) hour. A 1 M Tris-HCl (Sigma-Aldrich®, St. Louis, Mo.) (pH 8.0) portion was subsequently added (5% v/v) to stop the crosslinking reactions. Aliquots of the reactions and controls (conditioned media with no crosslinker added) were run on a 4-12% SDS-PAGE gel (Thermo Fisher Scientific®, Waltham, Mass.) followed by transfer to PVDF membrane (Millipore Sigma®, Burlington, Mass.) using the iBlot technology (Invitrogen/Thermo Fisher Scientific®, Carlsbad, Calif.). The membranes were bathed in TBS/Tween20 (0.1%, v/v) (Sigma-Aldrich®, St. Louis, Mo.) supplemented with 5% milk (w/v) and left for one (1) hour at RT. Subsequently, the membranes were sequentially exposed to goat anti-PGRN antibodies (AF2420 1:500) (R&D Systems®, Minneapolis, Minn.)) and rabbit anti-PSAP antibodies (HPA004426 1:200, HPA). Labeling of either antibody was detected with HRP-conjugated anti-Goat and anti-Rb antibodies, respectively, and HRP activity detected with a luminescence substrate (Pierce™ ECL Western Scientific Blotting Substrate (Thermo Fisher Scientific®, Waltham, Mass.) or Luminata™ Forte ELISA HRP Substrate (MilliporeSigma®, Burlington, Mass.)). The data supports that all cell lines express PSAP whereas PGRN could only be detected in cells stably expressing PGRN. Moreover, high molecular weight bands migrating at the same level in the gel, labelled with both anti-PGRN and anti-PSAP antibodies, are only detected in ARPE-PGRN and ARPE-PGRN+PSAP+scFv81 cells. Accordingly, PGRN/PSAP complexes are formed and secreted from ARPE cells stably expressing PGRN or both PGRN and PSAP.

### Example 15: Secreted Therapeutic Factors of Encapsulated ARPE-PGRN, ARPE-PSAP and ARPE-PGRN+PSAP+scFv81 Overexpressing Cells

The aforementioned cell lines were encapsulated according to a method previously described; from herein the encapsulated cell lines are denoted PGRN-, PSAP- and PGRN+PSAP devices, respectively. All devices were cultured in Gibco HE-SFM medium (Thermo Fisher Scientific®, Waltham, Mass.) at 37° C., 5% CO₂ for extended time periods, ranging from two (2) weeks to five (5) months. Aliquots of conditioned medium were analyzed for PGRN, PSAP and PGRN+PSAP secretion. PGRN-devices secrete both PGRN and PGRN/PSAP complexes, PSAP-devices secrete only PSAP and PGRN+PSAP devices secrete only PGRN/PSAP complexes (FIG. 3).

### Example 16: Activity of Secreted Factors from ARPE-PGRN, ARPE-PSAP and ARPE-PGRN+PSAP Cell Lines; Targeting of Cortical Neurons

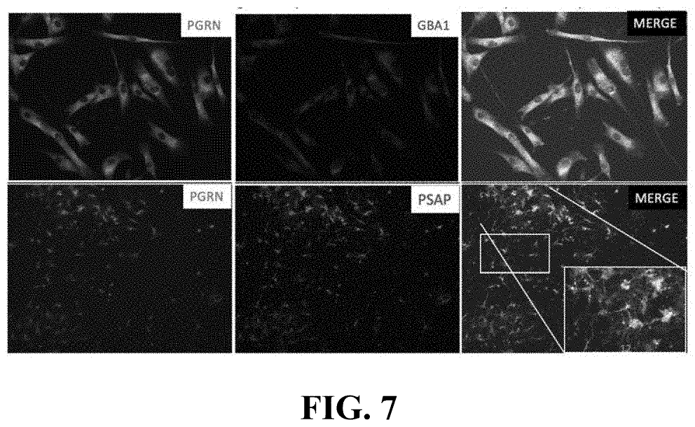

Cells were grown to confluency in 225 cm² tissue culture dishes in complete media. The cell media was then replaced with Gibco® FreeStyle™ 293 Expression Medium (Invitrogen/Thermo Fisher Scientific®, Carlsbad, Calif.) and the cells were cultured for seventy-two (72) hours at 37° C., 5% CO₂. Media was subsequently recovered and concentrated using Amicon® Ultra-4 Centrifugal filters (Millipore Sigma®, Burlington, Mass.) cut-off 30 kDa. Mouse primary cortical neurons, prepared from embryonic day seventeen (17) and cultured for twelve to fourteen (12-14) days (Div 12-14) at 37° C., 5% CO₂, were exposed to the concentrated conditioned media so the final [PGRN], [PSAP] or [PGRN/PSAP] was 1 μg/ml. The reactions were left overnight and the cells were subsequently fixed and subjected to immunocytochemical analysis using antibodies specific for different lysosomal markers, including PGRN, PSAP and LAMP1. FIG. 7 shows targeting of cortical neurons by the PGRN/PSAP complex that shows a very strong interaction to cortical neurons.

### Example 17: Activity of Secreted Factors from ARPE-PGRN, ARPE-PSAP and ARPE-PGRN+PSAP Cell Lines; Stimulation of GBA1 Activity

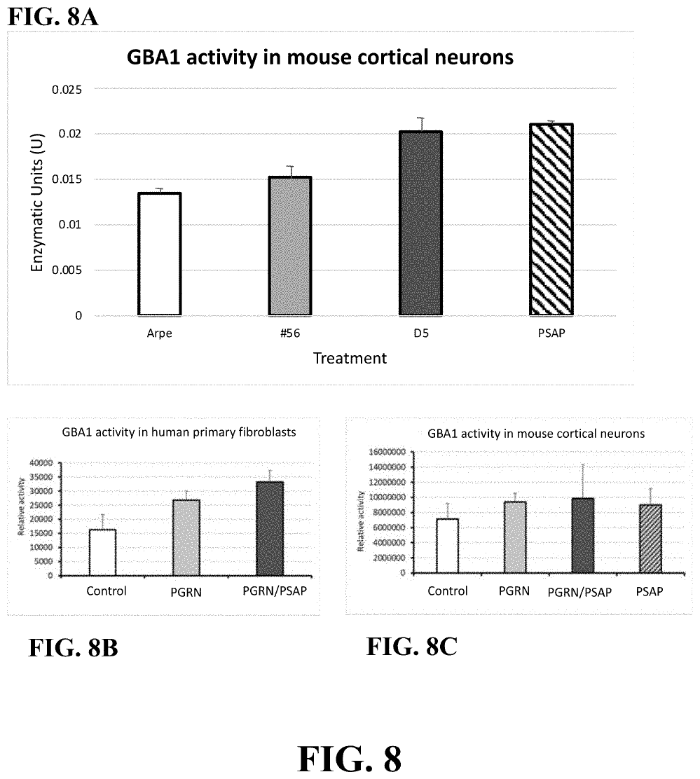

The different ARPE-cell lines were grown to confluency in 225 cm² tissue culture dishes in complete media. The cell media was then replaced with Gibco® FreeStyle™ 293 Expression Medium (Invitrogen/Thermo Fisher Scientific®, Carlsbad, Calif.) and the cells were cultured for another seventy-two (72) hours at 37° C., 5% CO₂. The conditioned media was then recovered and concentrated using Amicon® Ultra-4 Centrifugal filters (Millipore Sigma®, Burlington, Mass.) cut-off 30 kDa. Primary mouse cortical neurons, Div 12-14, were subsequently exposed to the concentrated conditioned media at 1 μg/ml final [PGRN] and [PGRN/PSAP]. The reactions were left for twenty (24) hours at 37° C., 5% CO₂, and then terminated by discarding the media from the Nunc MaxiSorp™ 96-well plates (cat no. 442404 (Thermo Fisher Scientific®, Waltham, Mass.)) followed by addition of activity buffer NaCitrate™ ((trisodium citrate dihydrate) (Sigma-Aldrich®, St. Louis, Mo.) (pH 5.4)), Triton™ X-100 (Sigma-Aldrich®, St. Louis, Mo.) (0.25% (v/v)), Taurocholic acid (2-{[(3α,5β,7α,12α)-3,7,12-trihydroxy-24-oxocholan-24-yl]amino}ethanesulfonic acid) (Sigma-Aldrich®, St. Louis, Mo.) (0.25% (w/v)) and 1 mM EDTA (2,2′,2″,2″′-(Ethane-1,2-diyldinitrilo)tetraacetic acid) (Sigma-Aldrich®, St. Louis, Mo.) after which the plates were immediately put in −85° C. to allow efficient lysis of the cells. To monitor GBA1 activity, the lysates were first thawed and incubated on ice for twenty (20) minutes before centrifugation at 4° C. for twenty (20) minutes at 20000 RCF to remove cell debris. The supernatants were collected and divided into two aliquots to test for GBA1 activity and to determine protein concentration, respectively. To test for GBA1 activity, the lysates were mixed with 1% BSA (Alfa Aesar®, Tewksbury, Mass.), 1 mM 4-Methylumbelliferyl b-glucophyranoside ((4-MU) (#M3633) (Sigma-Aldrich®, St. Louis, Mo.)) in 50 μl volume and then incubated at 37° C. for forty (40) minutes. The reactions were stopped with 1 volume of 1 M glycine (Sigma-Aldrich®, St. Louis, Mo.), pH 12.5, and the fluorescence monitored (ex=355 nm, em=460 nm) using a SpectraMax® D5 Series Multi-Mode Microplate Reader (Molecular Devices®, San Jose, Calif.). FIG. 8 shows that treatment with conditioned media from ARPE-PGRN and ARPE-PGRN/PSAP cells as well as recombinant PSAP result in increased GBA1 activity in differentiated mouse primary cortical neurons.

### Example 18: In vivo Functionality of PGRN-, PSAP- and PGRN+PSAP+scFv81 Devices

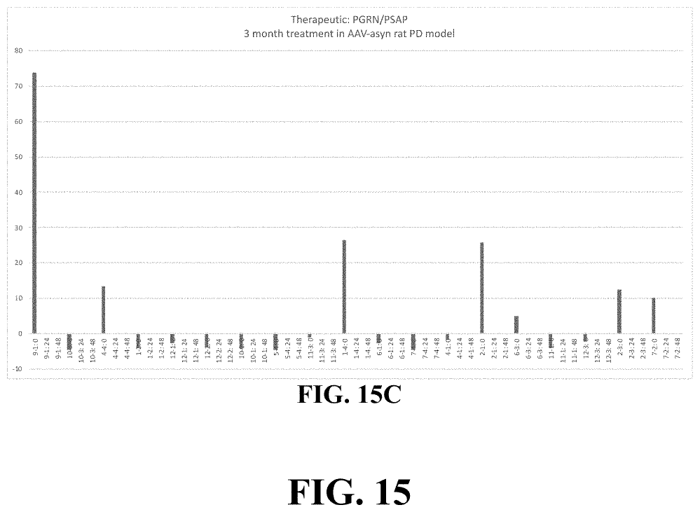

The in vivo functionality of the devices was tested by striatal implantation in rats in a manner similar to the study outline previously described (Tornøe J et al., (2012), Restor Neurol Neurosci, 30(3):225-36). Rats were treated for four to twenty-four (4 to 24) weeks and the devices were then removed for functionality testing by monitoring their PGRN, PSAP and PGRN/PSAP complex release. FIG. 15 shows functioning of all three types of devices after twelve (12) weeks treatment in the rat. In a next series of experiments, the in vivo functionality of PGRN-, PSAP- and PGRN+PSAP+scFv81 devices were explored after intrastriatal and intracerebroventricular (ICV) placement in the pig of clinical devices. After two to three (2 to 3) weeks of treatment of either therapy was tested, the devices were recovered and their production of secreted factors was assessed.

### Example 19: In vivo Safety of PGRN-Devices

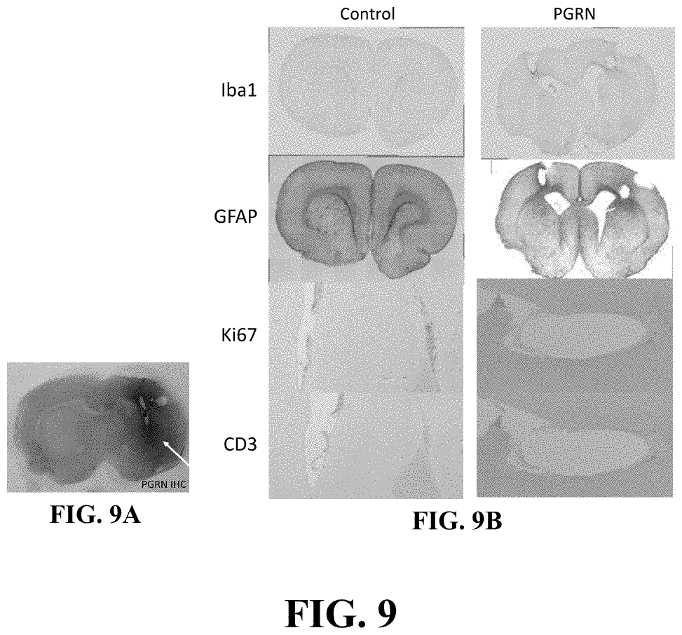

Sprague Dawley® native rats (Charles River Laboratories, Wilmington, Mass.), treated for twenty-four (24) weeks with PGRN-devices, were sacrificed and the brains recovered and subjected to fixation and paraffin embedding (ABCAM® PLC Co., Cambridge, UK) for histopathological assessment. Coronal sections (5 um) were incubated with antibodies raised against human PGRN, Ki67, GFAP, Iba1 and CD3 to monitor exposure, proliferating cells, inflammatory reactions and infiltrating T-cells, respectively. As shown in FIG. 9, twenty-four (24) weeks of treatment with PGRN-devices resulted in broad PGRN distribution in the brain, but no signals, other than what was expected due to the neurosurgical procedure itself (limited astrogliosis as determined by an increase in GFAP- and Iba-like immunoreactivity in the vicinity to where the devices were located), were observed.

### Example 20: PGRN-, PSAP- and PGRN+PSAP-Device Treatments Show Therapeutic Activity in 2 Different Rat Models of Neurodegeneration

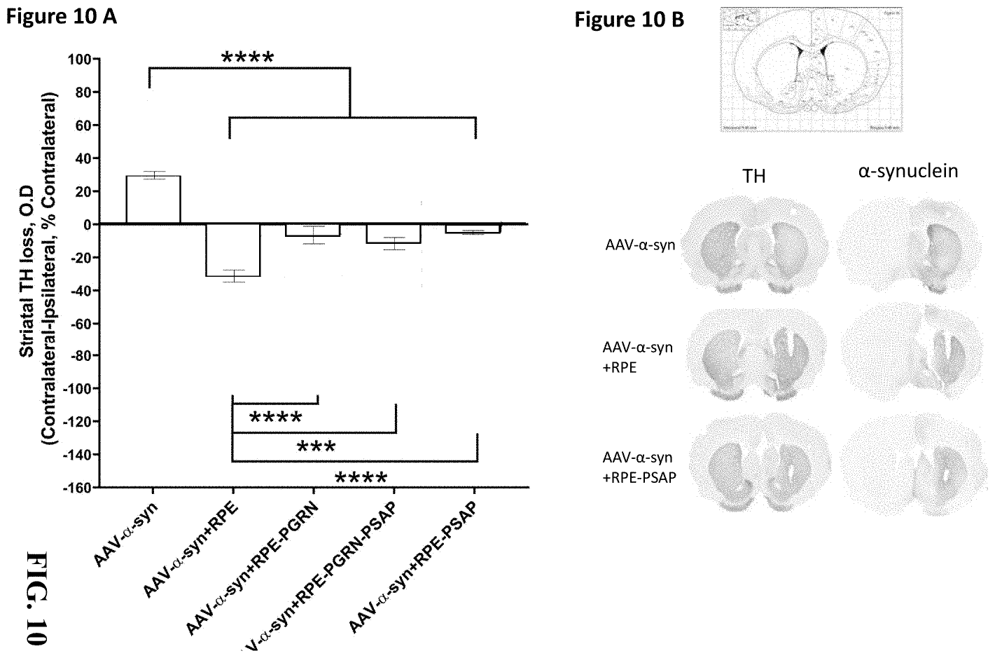

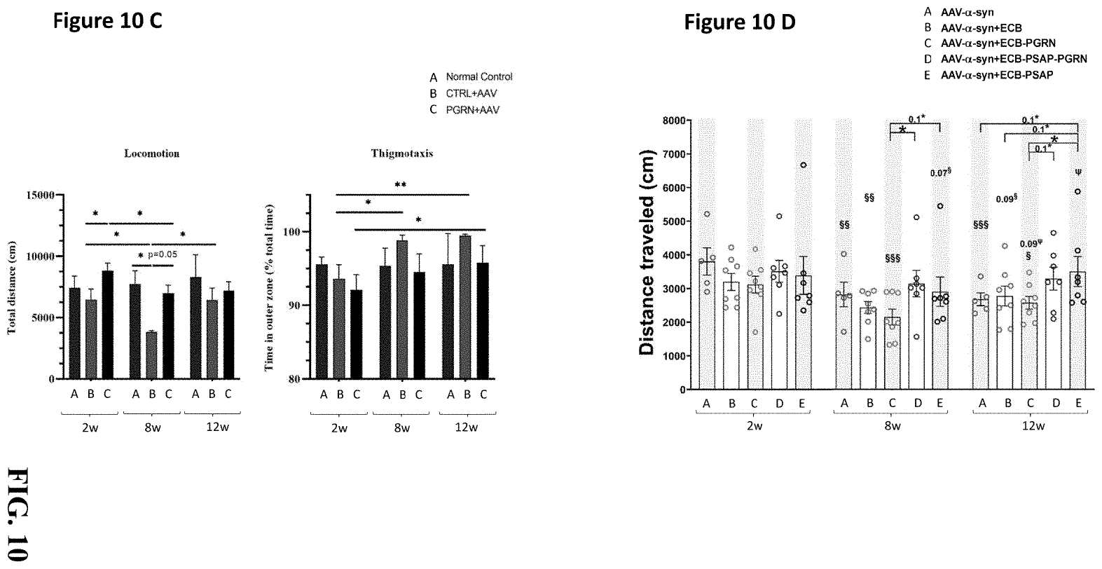

PGRN-, PSAP- and PGRN+PSAP-scFv81 devices were implanted in the striatum of rats that also got an injection in substantia nigra of AAV9 virus carrying a human alpha-synuclein gene (Decressac M et al., (2012), Neurobio Dis, 45(3):939-953). The rats were subjected to behavioral testing four, eight and twelve (4, 8, and 12) weeks post device implantation/virus injection. The rats were then sacrificed and the brains recovered for histopathological assessment (FIG. 10).

PGRN-, PSAP- and PGRN+PSAP-devices were implanted in the striatum of rats as described before (Tornøe J et al., (2012), Restor Neurol Neurosci, 30(3):225-36). A week post-surgery, the rats were subjected to behavioral testing (FIG. 11). The day after behavioral testing, the rats were unilaterally injected with 6-OHDA in substantia nigra compacta in order to trigger a PD-like neurodegenerative cascade. Approximately, two (2) and five (5) weeks post the 6-hydroxydopamine (6-OHDA) (Santa Cruz Biotechnology, Inc., Dallas, Tex.) injection, the rats were subjected to the same behavior paradigms as described above. The rats were then sacrificed and the brains recovered for histopathological assessment. PGRN-, and PGRN+PSAP-device treatments show improvement in the behavioral contexts assessed (FIG. 11).

### Example 21: GDNF+PGRN Secreting Devices, GDNF+PSAP Secreting Devices and GDNF+PGRN+PSAP Secreting Devices

GDNF+PGRN secreting devices, GDNF+PSAP secreting devices and GDNF+PGRN+PSAP secreting devices show therapeutic activity in the rat 6-OHDA model of neurodegeneration. Devices filled with ARPE-GDNF and either of the ARPE-factor cell lines described in Example 12 above, were tested for therapeutic activity as described in Example 12. All treatments showed therapeutic activity (FIG. 11).

### Example 22: Cell Culture Method for Production of Recombinant PGRN/PSAP Complexes

ARPE-19-PGRN+PSAP clone #D5 cells were cultured in complete media. Three flasks (225 cm²) were trypsinated (TrypLE™ Express Enzyme, Gibco®, 12605-010 (Thermo Fisher Scientific®, Waltham, Mass.)) and the cells were resuspended in 550 ml complete media and then seeded in a Corning® HYPERFlask® M Cell Culture Vessel (1720 cm² area)(Sigma-Aldrich®, St. Louis, Mo.). Three (3) days post seeding, the media was removed and the cells were washed with 2×100 ml PBS (HyClone® Laboratories, Inc., South Logan, Utah). Then, 550 ml Gibco® FreeStyle™ 293 Expression Media (Thermo Fisher Scientific®, Waltham, Mass.) supplemented with 1x Gibco® Penicillin-Streptomycin (PEST) (Thermo Fisher Scientific®, Waltham, Mass.), Geneticin® (G418) (Life Technologies® Corporation, Carlsbad, Calif.) and Hygromycin™ (Sigma-Aldrich®, St. Louis, Mo.) were added. The cells were cultured for ninety-six (96) hours at 37° C., 5% CO². The conditioned media was collected and replaced with 550 ml fresh Gibco® FreeStyle™ 293 Expression Media (Thermo Fisher Scientific®, Waltham, Mass.), supplemented with 1x Gibco® Penicillin-Streptomycin (PEST) (Thermo Fisher Scientific®, Waltham, Mass.), Geneticin® (G418) (Life Technologies® Corporation, Carlsbad, Calif.) and Hygromycin™ (Sigma-Aldrich®, St. Louis, Mo.). The collected conditioned media was immediately frozen at −85° C. for later further protein purification.

### Example 23: Harvesting and Concentration of Conditioned Media Containing PGRN/PSAP Complexes

Frozen batches of cell culture media were slowly thawed overnight at 4° C. The media was then centrifuged at 7200 rcf for twenty (20) minutes to pellet dead cells and debris. To secure the complete removal of particles, the supernatant was sterile filtered/degassed using Sarstedt® Filtration Units (0.22 um filters, ref 83.3941.101)(Sarstedt® AG & Co., Nuembrecht, Del.) prior to further processing. Subsequently, the filtered conditioned media was concentrated ten (10) times using Amicon® Cell Filter stir technology (cat no. UFSC40001) (Millipore Sigma®, Burlington, Mass.) loaded with a 30 kDa cut-off filter. The resulting concentrate was then subjected to different chromatographic methods.

### Example 24: Purification of PGRN/PSAP Complexes

Two different chromatographic methods to purify PGRN/PSAP complexes were applied: ion exchange chromatography and size exclusion chromatography (SEC), respectively. First, the concentrated sterile filtered media was run on a 6 ml Diethylaminoethyl cellulose (DEAE) column (Waters™ Technology Corporation, Milford, Mass.) and eluted with a 0-50% gradient 2M NaCl solution. Fractions were subsequently analyzed with SDS-PAGE gel (Thermo Fisher Scientific®, Waltham, Mass.) and PGRN/PSAP complex assay. PGRN/PSAP complexes were identified in three (3) fractions that were pooled and further subjected for SEC.

To ensure efficient separation, a HiLoad® 26/60 Superdex® 200 PG column (GE Healthcare Process R&D AB, Uppsala, SE) was used. Fractions containing PGRN/PSAP complexes were identified using three (3) different assays: PSAP ELISA (& Example 5), PGRN ELISA (hPGRN ELISA DuoSet® kit (#DY2420) (R&D Systems®, Minneapolis, Minn.)) and a PGRN/PSAP assay (& Example 5). Aliquots from each fraction were analyzed by these assays as well as by SDS-PAGE analysis (Thermo Fisher Scientific®, Waltham, Mass.). PGRN/PSAP complexes eluate in fractions different from free PGRN (FIG. 4). In addition, western blot analysis suggests that the stoichiometry of PGRN and PSAP is 1:1 the PGRN/PSAP complexes. Fractions containing only PGRN/PSAP complexes were pooled and recovered.

### Example 25: Storage of PGRN/PSAP

The pooled fractions with PGRN/PSAP complexes were dialyzed overnight in sterile PBS solution (HyClone® Laboratories, Inc., South Logan, Utah), aliquoted in sterile polypropylene Eppendorf® Tubes (Eppendorf® AG, Hamburg, Del.), snap frozen, and stored at −85° C.

### Example 26: Activity of Purified PGRN/PSAP Complexes; Targeting of Cortical Neurons

Extracellularly administered PGRN/PSAP interacts efficiently with mouse cortical primary neurons, is internalized and targets the lysosome. Mouse primary cortical neurons, prepared from embryonic day seventeen (17) were cultured at 37° C., 5% CO² for fourteen (14) days in BD Falcon™ 96-well cell culture dishes (BD Biosciences, Bedford, Mass.) prior to treatment. Sampled of PGRN, PSAP or PGRN/PSAP concentrated to 1 to 5 μg/ml were then added and the cultures incubated at 37° C., 5% CO². The media was subsequently removed and the cells fixed and subjected to immunocytochemical analysis using antibodies specific for different lysosomal markers, including PGRN, PSAP and GBA1. As evidenced by FIG. 7, efficient targeting of cortical neurons by the PGRN/PSAP complex that colocalizes with GBA1 suggesting that the complex is internalized and targets the lysosome.

### Example 27: Activity of Purified PGRN/PSAP Complexes. Stimulation of GBA1 Activity

An extracellularly administered PGRN/PSAP complex, PGRN or PSAP colocalize with, and each treatment activate, GBA1 in mouse primary cortical neurons and human primary fibroblasts, derived from heterozygous GBA1 L444P mutation carriers. Mouse primary cortical neurons prepared from embryonic day seventeen (17) were cultured at 37° C., 5% CO² for fourteen (14) days in BD Falcon™ 96-well cell culture dishes (BD Biosciences, Bedford, Mass.) prior to treatment. PGRN, PSAP or PGRN/PSAP were then added to a concentration of 10 ng/ml and cultures were incubated for twenty-four (24) hours prior to analysis. Human fibroblasts were grown to confluency and then treated with PGRN, PSAP or PGRN/PSAP GBA1 as aforementioned described for the mouse primary neuronal cultures. After twenty-four (24) hours of treatment at 37° C., 5% CO², the reactions were terminated by removing the media from the Nunc MaxiSorp™ 96-well plates and adding an activity buffer consisting of NaCitrate™ ((trisodium citrate dihydrate) (pH 5.4) (Sigma-Aldrich®, St. Louis, Mo.)), Triton™ X-100 ((0.25% (v/v)) (Sigma-Aldrich®, St. Louis, Mo.)), taurocholic acid ((2-{[(3α,5β,7α,12α)-3,7,12-trihydroxy-24-oxocholan-24-yl]amino}ethanesulfonic acid) (0.25% (w/v) (Sigma-Aldrich®, St. Louis, Mo.)) and 1 mM EDTA ((2,2′,2″,2″′-(Ethane-1,2-diyldinitrilo)tetraacetic acid) (Sigma-Aldrich®, St. Louis, Mo.)) were added and the plates were put in −85° C. to allow efficient lysis and brake of cells. To monitor GBA1 activity, the lysates were first thawed and incubated on ice for twenty (20) minutes before centrifugation at 4° C. for twenty (20) minutes at 20000 rcf to remove cell debris. The supernatants were collected and divided into two (2) aliquots for GBA1 activity and protein concentration determination, respectively. For GBA1 activity, the lysates were mixed with 1% BSA (Alfa Aesar®, Tewksbury, Mass.), 1 mM 4-Methylumbelliferyl b-glucophyranoside (4-MU, Sigma-Aldrich®, #M3633) in 50 μl volume and then incubated at 37° C. for forty (40) minutes. The reactions were stopped with one (1) volume of 1 M glycine at pH 12.5 and the fluorescence was monitored (ex=355 nm, em=460 nm) using a Spectramax® D5 plate reader (Molecular Devices®, San Jose, Calif.). FIG. 8 shows that treatment with PGRN, PSAP and PGRN/PSAP complexes increase GBA1 activity in differentiated mouse primary cortical neurons human primary fibroblasts heterozygous for a loss of function GBA1 mutation.

### Example 28: Activity of Purified PGRN/PSAP Complexes. Stimulation of Neurite Outgrowth

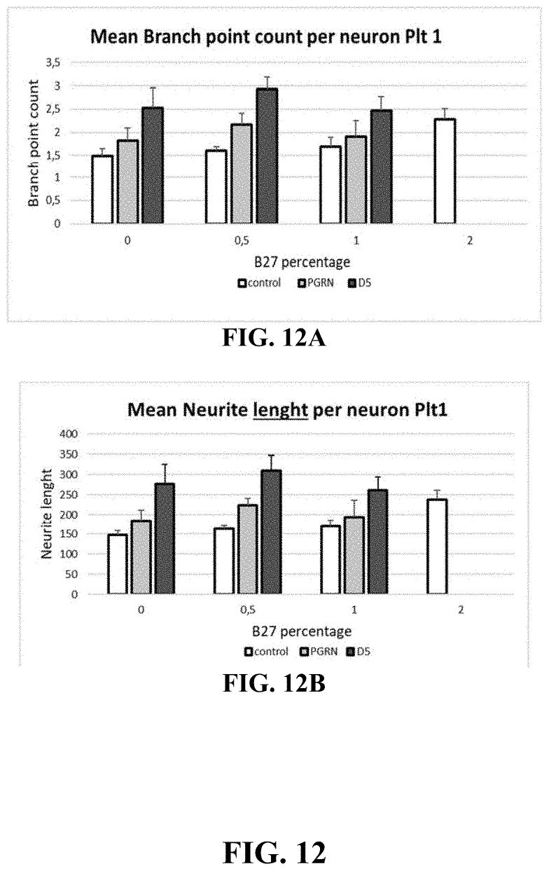

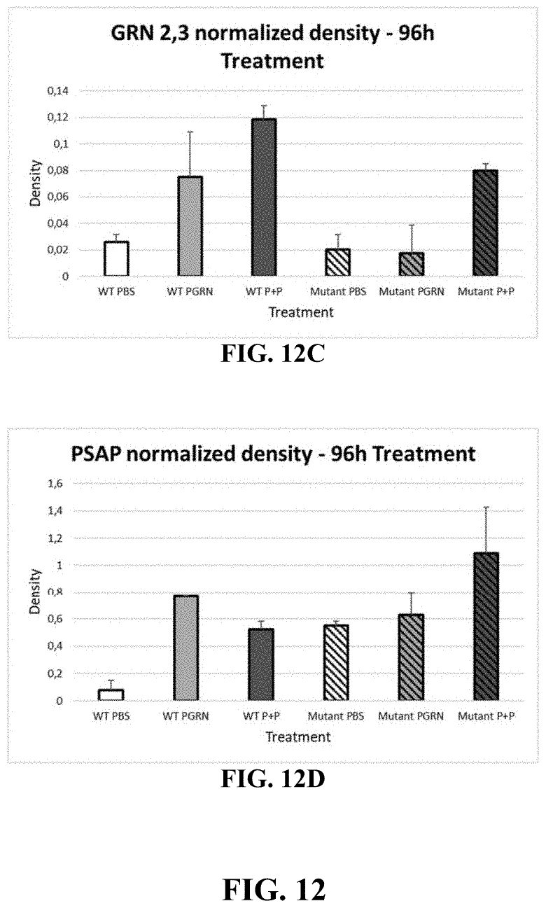

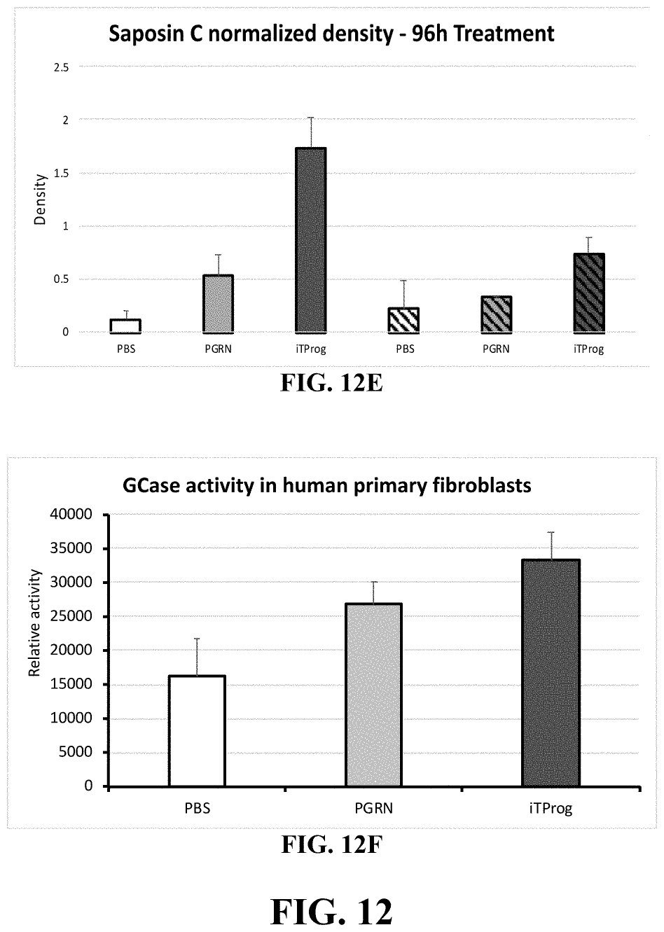

Extracellularly administered PGRN/PSAP stimulates neurite outgrowth in mouse primary cortical neuronal cultures. Mouse primary cortical neurons were prepared from embryonic day seventeen (17). Brains were harvested and cortical cultures prepared according to known methods. (Merino-Serrais P et al., (2019), Cereb Cortex, 29(1):429-46). Cells were seeded in BD Falcon™ 96-well Poly-L-coated cell culture dishes (BD Falcon™ 96-well cell culture dishes (BD Biosciences, Bedford, Mass.)) in neurobasal media (Neurobasal™ Plus Medium, (Gibco®, Life Technologies®, Carlsbad, Calif.), supplemented with L-Glutamine, Gibco® Penicillin-Streptomycin (PEST) (Thermo Fisher Scientific®, Waltham, Mass.) and 2% B27 (Anti-Human Leukocyte Antigen B27 antibody (ABCAM® PLC Co., Cambridge, UK). Six (6) hours post seeding, the media was removed and directly replaced with 90 μl/well neurobasal media (Neurobasal™ Plus Medium, (Gibco®, Life Technologies®, Carlsbad, Calif.)), supplemented with L-Glutamine, Gibco® Penicillin-Streptomycin (PEST) (Thermo Fisher Scientific®, Waltham, Mass.) and either 0%, 0.5 or 1% B27 (Anti-Human Leukocyte Antigen B27 antibody (ABCAM® PLC Co., Cambridge, UK). Cultures with complete media, i.e. 2% B27 (Anti-Human Leukocyte Antigen B27 antibody (ABCAM® PLC Co., Cambridge, UK), served as control. Ten (10) μl neurobasal media (Neurobasal™ Plus Medium, (Gibco®, Life Technologies®, Carlsbad, Calif.)), supplemented with L-Glutamine and Gibco® Penicillin-Streptomycin (PEST) (Thermo Fisher Scientific®, Waltham, Mass.) and 10 ng/ml of either PGRN or PGRN/PSAP were then added and the cultures were further incubated at 37° C. for four (4) days or, approximately ninety-six (96) hours. Media was then discarded and the cells fixed in 4% formaldehyde (O═CH₂) (Fisher Chemical, Waltham, Mass.) for thirty (30) minutes at room temp. The fixation solution was subsequently discarded and the wells washed three (3) times with PBS (HyClone® Laboratories, Inc., South Logan, Utah) prior to immunocytochemical (ICC) analysis: fixed cells were first treated for one (1) hour at room temperature with PBS (HyClone® Laboratories, Inc., South Logan, Utah)/ Triton™ X-100 (t-Oct-C₆H₄-(OCH₂CH₂)ₓOH, x=9-10, (MilliporeSigma®, Burlington, Mass.)) (0.25%) and BSA (Alfa Aesar®, Tewksbury, Mass.) (3%) to permeabilize the cells and to block nonspecific protein binding. After removing the blocking solution, the cultures were exposed to fresh blocking solution supplemented with mouse monoclonal anti-Tubulin antibodies ((anta Cruz, G8) (ABCAM® PLC Co., Cambridge, UK)), diluted to 1:200, and then incubated on at +4° C. Subsequently, cells were washed three (3) times in with PBS (HyClone® Laboratories, Inc., South Logan, Utah)/ Triton™ X-100 (MilliporeSigma®, Burlington, Mass.) (0.25%) and BSA (Alfa Aesar®, Tewksbury, Mass.) (3%) and then incubated for one (1) hour at room temperature with a blocking solution supplemented with Alexa594 goat anti-mouse IgG (1:1000) (Alexa Fluor™, Thermo Fisher Scientific®, Waltham, Mass.) and 10 ug/ul bisBenzimide blue fluorescent dye, i.e. Hoechst stain (Höchst, Frankfurt, Del.). After removal of the reaction mixture, the BD Falcon™ 96-well plates were washed three (3) times in PBS/Triton™ x-100, i.e. PBS (HyClone® Laboratories, Inc., South Logan, Utah)/ Triton X-100 (MilliporeSigma®, Burlington, Mass.) and then stored in a dark environment at +4° C. prior to analysis. Neuronal morphology was monitored using a High-Content Screening Platform (HCS) part of Cellomics™ technology (ArrayScan™, Thermo Fisher Scientific®, Waltham, Mass.) and the neurite morphology software (Data61®, CSIRO, Canberra, AU). PGRN/PSAP, like PGRN, stimulates neurite outgrowth as shown in FIG. 12.

### Example 29: Activity of Purified PGRN/PSAP Complexes. Distribution into the Brain, Internalization and Lysosomal Targeting after Intracerebroventricular (ICV) Administration

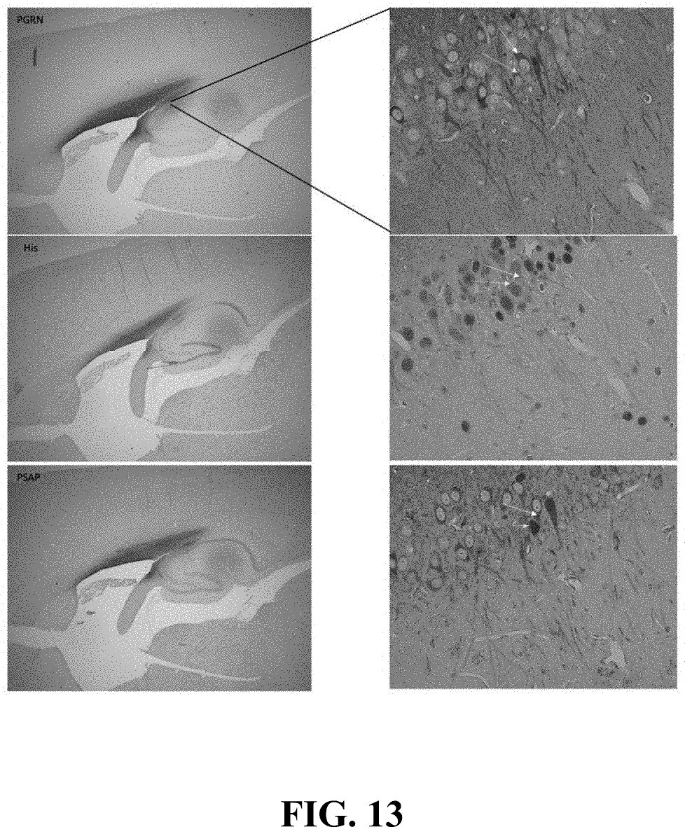

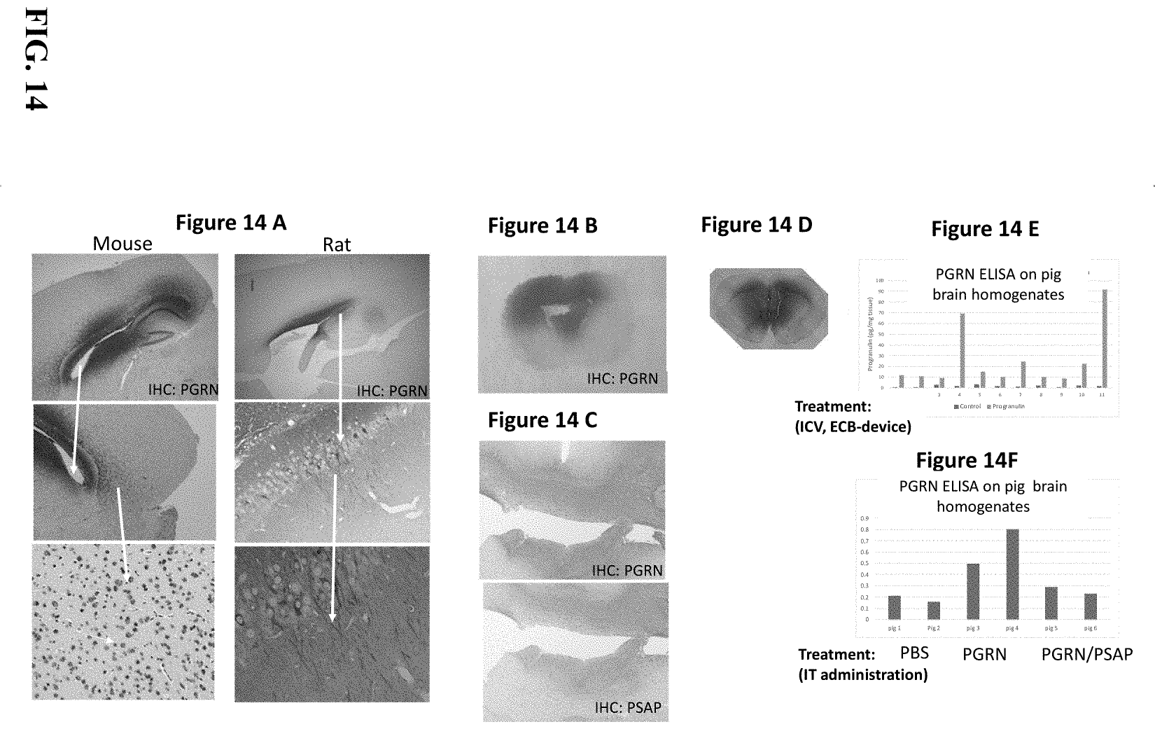

Conditioned media from ARPE-PGRN and ARPE-PSAP cells were prepared, concentrated and buffer exchanged to PBS. Recombinant PGRN-His (cat no. 2420-PG (R&D Systems®, Minneapolis, Minn.)) was mixed with the concentrated conditioned media from ARPE-PSAP cells and PGRN/PSAP complexes were formed as demonstrated by western blot analysis FIG. 2. ARPE-derived PGRN and PGRN/PSAP complexes from ARPE-PGRN cells were identified with western blot analysis (FIG. 2). The reaction mixtures, prepared in PBS, were administered to the test subject by intracerebroventricular (ICV) injection (Hamilton® Co., Reno, Nev.) using a peristaltic pump (Thermo Fisher Scientific®, Waltham, Mass.) at a rate of 0.5 μl per minute for approximately two (2) minutes. The mice were sacrificed three (3) hours post ICV injection and the brains were recovered, put in fixative (4% formaldehyde in PBS) for forty-eight (48) hours and then stored in a solution comprising PBS/30% sucrose at a temperature of +4° C. until needed. The brains were subsequently paraffin embedded (Weiss AThA et al., (2011), Vet Pathol, 48(4):834-8) prior to immunohistochemical analysis: Human PGRN- and PSAP-like immunoreactivity were monitored using the following PGRN antibodies: MAB2420 and AF2420 (R&D Systems®, Minneapolis, Minn.), Penta-His (anti-his tag directed antibody (Qiagen, Venlo, NL)) and PSAP antibodies (Abnova® cat no. H00005660-M01, 0.43 mg/ml (Abnova® GmbH, Taipei, TW) and (Proteintech® cat no. HPA004426, 0.1 mg/ml (Proteintech®, Rosemont, Ill.)). After incubations with the appropriate HRP-conjugated secondary antibodies, PGRN- and PSAP-like and His-like immunoreactivity were detected with DAB as substrate for HRP. FIG. 13 shows overlapping diffusion into the brain of PGRN and PSAP from mice injected with concentrated conditioned media from ARPE-PGRN cells and ARPE-PSAP conditioned media mixed with PGRN-His. At higher magnification, intracellular colocalization of PGRN and PSAP were detected in these brains and also intracellular colocalization of His-like and PSAP-like immunoreactivity, suggesting that ICV administered PGRN/PSAP complexes are diffused into the brain and internalized by brain cells. FIG. 14 shows that recombinant PGRN-Fc, PGRN and PGRN/PSAP complexes, purified as described in Example 23, are taken up and broadly distributed in the rat brain. In addition, purified PGRN administered via a catheter IT in the pig and ICV ECB-PGRN treatment for two weeks in the pig, results in brain uptake of PGRN. Thus, IT and ICV delivery of PGRN and PGRN/PSAP diffuse into the mouse, rat and pig brains.

### Definitions

For convenience, certain terms employed in the specification, examples and appended claims are collected here. These definitions should be read in light of the disclosure and understood by a person of ordinary skill in the art.

As used herein, the term “bioreactor” refers to any manufactured device or system that supports a biologically active environment. In various embodiments, a bioreactor is a vessel in which a chemical process is carried out which involves organisms or biochemically active substances derived from such organisms. This process can either be aerobic or anaerobic. In a further embodiment, a bioreactor may also refer to a device or system designed to grow cells or tissues in the context of cell culture. In yet a further embodiment, molecules secreted or produced by the cells grown in a bioreactor may be harvested and purified.

As used herein, the terms “capsule,” or alternatively, “encapsulate” or “encapsulated,” refer to an enclosed device (or method of using said device) containing cells, preferably by a semi-permeable membrane that permits the bidirectional diffusion of molecules such as the influx of oxygen, nutrients, growth factors etc., essential for cell metabolism and the outward diffusion of waste products and therapeutic proteins. At the same time, the semi-permeable nature of the membrane prevents immune cells and antibodies from destroying the encapsulated cells regarding them as foreign invaders.

As used herein, the term “cell line” refers to a population of cells derived from a single progenitor cell that can be propagated repeatedly or indefinitely. The progenitor cell may be derived from the organ or tissue of a larger animal or plant.

As used herein, the terms “expression,” or alternatively, “express, ” “expressing, ” “expressed” or “to express, ” refer to the transcription and stable accumulation of sense RNA (mRNA) or antisense RNA derived from a nucleic acid or polynucleotide. Expression may also refer to translation of mRNA into a protein or polypeptide.

As used herein, the term “expression construct” refers to any molecule, virus, or organism designed to introduce a nucleic acid or polynucleotide into a cell for the purpose of expressing a protein or RNA encoded by that nucleic acid or polynucleotide. In a preferred embodiment, an expression construct may be a plasmid. An expression construct may also refer to an expression vector, and these terms are used interchangeably.

As used herein, the terms “fragment,” or alternatively, “a fragment thereof,” when applied to a polynucleotide sequence refer to a nucleotide sequence comprising the same nucleotide sequence as the reference nucleic acid over a common portion with a reduced length relative to the reference nucleic acid. Such a nucleic acid fragment according to the invention may be contained in a larger polynucleotide, if appropriate, which is a constituent thereof.

As used herein, the terms “heterodimer,” or alternatively, “heterodimers” or “heterodimerization,” refer to a macromolecular complex formed by two protein monomers, or single proteins, wherein the two protein monomers comprise two different protein sequences.

As used herein, the term “immunoisolatory” refers to a method or means of protecting implanted material such as biopolymers, cells, or drug release carriers from an immune reaction or minimizing an immune reaction. In an embodiment, an implantable device may be immunoisolatory in that in protects material inside of the device from an immune reaction after the device is implanted in a host.

As used herein, the terms “implantable,” or alternatively, “implant,” “implants,” “implanted,” or “to implant, ” refer to a device designed to be introduced into the body of a host for an extended period of time, for the purpose of replacing, augmenting, or supporting an existing biological structure or function of the host.

As used herein, the term “matrix” refers to a three-dimensional network of extracellular macromolecules, such as polymers, collagen, enzymes, laminin, fibronectin, or glycoproteins, that provide structural and biochemical support to surrounding cells.

As used herein, the terms “modify,” or alternatively, “modified,” “modifies,” “modification,” “modifying” or “to modify, ” refer to any alteration of matter which, directly or indirectly, enhances, diminishes, adds, or removes a property or properties of said matter.

As used herein, the terms “neurological disease” and “neurological disorder” are used interchangeably and refer to any functional abnormality or disturbance of the nervous system, whether caused by structural, biochemical, or electrical abnormalities in the brain, spinal cord or other nerves.

As used herein, the term “other chromatographic methods” refers to any technique used for the separation of a mixture, whether preparative or analytical, as known in the art at the time of filing or discovered thereafter.

As used herein, the terms “precursor polypeptide,” “protein precursor,” or “pro-protein” are used interchangeably and refer to an inactive protein (or peptide) that can be turned into an active form by post-translational modification, such as breaking off a piece of the molecule or adding on another molecule.

As used herein, the terms “purified,” or alternatively, “purify,” “purified,” “purification” or “to purify” refer to a substance which has been substantially increased in concentration or freed of contaminants. This term does not necessarily indicate absolute purity unless otherwise indicated.

As used herein, the term “Sleeping beauty Transposase System” refers to a method of introducing DNA sequences into the genome of a cell by means of a Sleeping beauty transposase and a transposon, as well as materials to perform said method.

As used herein, the terms “subpeptide,” or alternatively, “subpeptides” or “subpeptides thereof” refer to a polypeptide that is derived from part of a larger protein or polypeptide. In an embodiment, the subpeptide may be a fragment of the larger protein or polypeptide.

As used herein, the terms “therapeutic,” or alternatively, “a therapeutic,” “a therapeutic drug,” “a therapeutic agent,” “therapy,” “therapies,” “a therapeutic regimen” or “a therapeutic method” refer to any molecule (or method using said molecule) that confers a beneficial function to the subject being treated with said molecule. Therapeutics may include, but are not limited to, peptides, polypeptides, single or multi-chain proteins, fusion proteins, antisense oligonucleotides, small interfering RNAs, ribozymes, and RNA external guide sequences. The therapeutic may include naturally occurring sequences, synthetic sequences, or a combination of natural and synthetic sequences.

As used herein, the terms “comprises,” “comprising,” “includes,” “including,” “has,” “having,” or any other variations thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, ‘or’ refers to an inclusive or and not to an exclusive or. For example, a condition A or B is satisfied by any one of the following: A is true (or present) and B is false (or not present), A is false (or not present) and B is true (or present), and both A and B are true (or present). Also, use of the terms “a” or “an” are employed to describe elements and components of the invention. This is done merely for convenience and to give a general sense of the invention. This description should be read to include one or at least one and the singular also includes the plural unless it is obvious that it is meant otherwise. Unless as otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are discussed above. All publications, patent applications, patents, and other references mentioned herein are incorporated by reference in their entirety. In case of conflict, the present specification, including definitions, will control. In addition, the materials, methods, and examples are illustrative only and not intended to be limiting. In the following description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout the specification are not necessarily all referring to the same embodiment. Furthermore, the particular features, structures, or characteristics may be combined in any suitable manner in one or more embodiments.

The term “and/or” as used herein is defined as the possibility of having one or the other or both. For example, “A and/or B” provides for the scenarios of having just A or just B or a combination of A and B. If the claim reads “A and/or B and/or C, ” the composition may include A alone, B alone, C alone, A and B but not C, B and C but not A, A and C but not B or all three A, B, and C components.

### Acronyms

For convenience, certain terms employed in the specification, examples and appended claims are collected here. These definitions should be read in light of the disclosure and understood as by a person of ordinary skill in the art.

| | |
|---|---|
| AAV | adeno associate virus vector |
| AD | Alzheimer's disease |
| ALS | amyotrophic lateral sclerosis |
| APRE-19 | adult retinal pigment epithelial cell line-19 |
| bFGF | basic fibroblast growth factor |
| BHK | baby hamster kidney cells |
| BMT | bone marrow transplant |
| BSA | bis(trimethylsilyl)acetamide |
| BSC | Cercopithecus monkey kidney cells |
| CAC | circulating angiogenic cells |
| CD3 | cluster of differentiation 3 |
| CHO | Chinese hamster ovary |
| CDNA1 | complementary DNA |
| CDNF | cerebral dopamine neurotrophic factor |
| CMV | (human) cytomegalovirus |
| CNS | central nervous system |
| COS | CV-1 (simian) in Origin |
| CSF | cerebrospinal fluid |
| DEAE | diethylaminoethyl cellulose |
| DMEM | Dulbecco's Modified Eagle Medium |
| ECB | encapsulated cell bio-delivery |
| EDTA | 2,2′,2″,2″′-(Ethane)-1,2- |
| | diyldinitrilo)tetracetic acid |
| EGF | epidermal growth factor |
| ELISA | enzyme-linked immunosorbent assay |
| FCS | fetal calf serum |
| bFGF | basic fibroblast growth factor |
| FITC | fluorescein isothiocyanate |
| FTD | frontotemporal dementia |
| FUS | fused in sarcoma |
| GBA1 | β-Glucocerebrosidase |
| GCase | glucocerebrosidase |
| GCFN | glial cell-derived neurotrophic factor |
| GDNF | glial cell-derived neurotrophic factor |
| | a/k/a ARMET-like protein 1 |
| GFAP | glial fibrillary acidic protein |
| GRN | granulin |
| HIV | human immune-deficiency virus |
| HRP | horseradish peroxidase |
| HEK | human embryonic kidney |
| Iba1 | ionized calcium-binding adapter molecule 1 |
| ICC | immunocytochemical |
| ICV | intracerebroventricular |
| iPS | induced pluripotent stem cells |
| IR/DR | inverted repeat/direct repeat elements |
| KEGG | Kyoto encyclopedia of genes and genomes |
| LAMP1 | lysosomal-associated membrane protein 1 a/k/a |
| | lysosome-associated membrane glycoprotein 1 a/k/a |
| | cluster of differentiation 107a |
| LATE | limbic-predominant age-related TAR DNA-binding |
| | protein-43 (TDP-43) encephalopathy |
| LB | Lewis body |
| LBD | Lewis body dementia |
| MANF | mesencephalic astrocyte-derived neurotrophic factor |
| MCS | mesenchymal chondrosarcoma |
| MSA | multiple system atrophy |
| MSC | mesenchymal stem cell |
| MSO | mesenchymal chondroSarcoma-1 |
| NCL | neuronal ceroid lipofuscinosis |
| NT | neurotrophin |
| NS0 | mouse myeloma cells |
| 6-OHDA | 6-hydroxydopamine |
| PBS | phosphated buffer solution |
| PC | pheochromocytoma (12 and 12A) |
| PD | Parkinson's disease |
| PEST | Penicillin/Streptomycin |
| PGRN | progranulin |
| PROTAC | proteolysis targeting chimera |
| PSAP | prosaposin |
| RCF | relative centrifugal force |
| RN | rat neuronal |
| RNA | ribonucleic acid |
| RPE | retinal pigment epithelium |
| RT | room temperature |
| SCC | squamous cell carcinoma |
| scFv | single chain variable fragment |
| SDS-PAGE | sodium dodecyl sulfate-polyacrylamide gel electrophoresis |
| SEC | size exclusion chromatography |
| SIRC | Startus Seruminstitut rabbit cornea |
| TAR | transactive response |
| TAT | trans-activator of transcription |
| TDP | TAR DNA-binding protein |
| hTERT | human telomerase reverse transcriptase |
| TH | tyrosine hydroxylase |
| TMB | 3,3′,5,5′-tetramethylbenzidine |

### Equivalents

The full scope of the invention should be determined by reference to the claims, along with their full scope of equivalents, and the specification, along with such variations.

Unless otherwise indicated, all numbers expressed quantities of ingredients, reaction conditions, and so forth use in the specification and claims are to be understood as being modified in all instances by the term “about,” defined as ±5%. Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

We claim:

1. A complex of progranulin and prosaposin.

2. The complex according to claim 1, wherein said complex is a heterodimer of progranulin and prosaposin.

3. The complex according to claim 1, wherein said complex is a fusion protein.

4. The complex according to claim 3, wherein said fusion protein is formed recombinantly.

5. The fusion protein according to claim 3, wherein the fusion protein comprises SEQ ID NO: 7, 9, 11, 13, 15, 17 or a fragment thereof.

6. A cell culture comprising a mammalian cell line which expresses or is modified to express a progranulin polypeptide and a prosaposin polypeptide.

7. The cell culture according to claim 6, wherein the mammalian cell line is genetically modified.

8. The cell culture according to claim 6, comprising a mammalian cell line which is modified to express subpeptides of progranulin and prosaposin precursor polypeptides.

9. The cell culture according to claim 6, wherein said progranulin is progranulin C-terminus for glial cell-derived neurotrophic factor (GCase) interaction.

10. A cell culture comprising a mammalian cell line which contains a gene expressing progranulin and a gene expressing prosaposin or is modified to contain a gene expressing progranulin and a gene expressing prosaposin.

11. The cell culture according to claim 10, wherein the progranulin gene is a cDNA.

12. The cell culture according to claim 10, wherein the prosaposin gene is a cDNA.

13. A cell culture comprising a mammalian cell line which expresses a gene for progranulin and a gene for prosaposin.

14. The cell culture according to claim 13, wherein the progranulin gene is a cDNA.

15. The cell culture according to claim 13, wherein the prosaposin gene is a cDNA.

16. The cell culture according to any one of claim 6 or 10, wherein the mammalian cell line is selected from the group consisting of: mouse myeloma cells (NS0), Chinese hamster ovary cells (CHO); Chinese hamster ovary cells (CHO)-K1; baby hamster kidney cells (BHK); mouse fibroblast-3T3 cells; African green monkey cell lines; mesenchymal chondroSarcoma-1 (MCS); rat adrenal pheochromocytoma (PC)-12; rat adrenal pheochromocytoma (PC)-12A; AT3, rat glial tumor (C6) cells; rat neuronal cell line RN33b; rat hippocampal cell line HiB5; growth factor expanded stem cells; epidermal growth factor (EGF)-responsive neurospheres; basic fibroblast growth factor (bFGF)-responsive neural progenitor stem cells derived from the central nervous system (CNS) of mammals; foetal cells; primary fibroblasts; Schwann cells; astrocytes; β-TC cells; Hep-G2 striatal cells; oligodendrocytes and their precursors; mouse myoblast cells-C2C12; human glial-derived cells-Hs683; human glial-derived cells-A172; HEI193T cell line; porcine glioblasts; neuronal cells; neurons; astrocytes; interneurons; chondroblasts isolated from human long bone; human embryonic kidney cells HEK293; human cell line HeLa; rabbit corneal-derived cells (Startus Seruminstitut Rabbit Conrnea cells (SIRC)); Human corneal derived cells, human choroid plexus cells, human induced pluripotent stem cells (iPS) derived cell lines, human neurotrophin 3 (NT3) cells, adult retinal pigments epithelial cell line-10 (ARPE-19), circulating angiogenic cells (CAC), immortalized human fibroblasts (MDX cells), telomerase immortalized human retinal pigment epithelium (RPE) cell lines and mesenchymal stem cells (MSC).

17. The cell culture according to claim 16, wherein the African green monkey cell lines are selected from the group consisting of COS-1, COS-7, SCC-1, BSC-40, BMT-10 and Vero cell lines.

18. The cell culture according to claim 16, wherein the human retinal pigment epithelium (RPE) cell line is human telomerase reverse transcriptase (hTERT) retinal pigment epithelium-1 (RPE-1).

19. The cell culture according to claim 16, wherein the preferred cell lines for mammalian recombinant production include ARPE-19, CHO, CHO-1, HEI193T, HEK293, COS, NS0, and BHK cells.

20. The cell culture according to any one of claim 6 or 10, wherein the progranulin polypeptide comprises SEQ ID NO: 2 or a fragment thereof.

21. The cell culture according to any one of claim 6 or 10, wherein the progranulin gene comprises SEQ ID NO: 1 or a fragment thereof.

22. The cell culture according to any one of claim 6 or 10, wherein the prosaposin polypeptide comprises SEQ ID NO: 4 or a fragment thereof.

23. The cell culture according to any one of claim 6 or 10, wherein the prosaposin gene comprises SEQ ID NO: 3 or 5 or fragments thereof.

24. The cell culture according to any one of claim 6 or 10, wherein the cell line comprises:
a first expression construct expressing progranulin, or
a second expression construct expressing prosaposin.

25. The cell culture according to claim 24, wherein the first expression construct comprises a plasmid.

26. The cell culture according to claim 24, wherein the second expression construct comprises a plasmid.

27. The cell culture according to claim 25, wherein the first expression construct further comprises a transposon system.

28. The cell culture according to claim 27, wherein the transposon system is a Sleeping beauty transposase system.

29. The cell culture according to claim 27, wherein the transposon system is a Piggy back transposase system.

30. The cell culture according to claim 24, wherein the second expression construct further comprises a transposon system.

31. The cell culture according to claim 30, wherein the transposon system is a Sleeping beauty transposase system.

32. The cell culture according to claim 30, wherein the transposon system is a Piggy back transposase system.

33. The cell culture according to claim 10, wherein the progranulin polypeptide comprises a progranulin-antibody fragment fusion protein or a gene that expresses a progranulin-antibody fragment fusion protein.

34. The cell culture according to claim 10, wherein the prosaposin comprises a prosaposin-antibody fragment fusion protein or a gene that expresses a prosaposin-antibody fragment fusion protein.

35. The cell culture according to any one of claim 33 or 34, wherein the antibody fragment of either the progranulin-antibody fragment or the prosaposin-antibody fragment increases the brain distribution and cellular uptake of progranulin, prosaposin, or a complex thereof.

36. The cell culture according to claim 10, wherein the progranulin gene expresses a progranulin-antibody fragment fusion gene.

37. The cell culture according to claim 10, wherein the prosaposin gene expresses a prosaposin-antibody fragment fusion.

38. The fusion gene according to any one of claim 36 or 37, wherein the fusion gene comprises SEQ ID NO: 6, 8, 10, 12, 14, 16 or a fragment thereof.

39. The cell culture according to any one of claim 36, 37 or 38, wherein the progranulin-antibody fragment fusion gene encodes a peptide sequence which increases the brain distribution and cellular uptake of progranulin, prosaposin, or a complex thereof; further wherein the prosaposin-antibody fragment fusion gene encodes a peptide sequence which increases the brain distribution and cellular uptake of progranulin, prosaposin, or a complex thereof.

40. The cell culture according to any one of claim 6 or 10, wherein the expressed progranulin and prosaposin form a complex before secretion from the cell.

41. The cell culture according to any one of claim 6 or 10, wherein the expressed progranulin and prosaposin form a complex after secretion from the cell.

42. The cell culture according to any one of claim 40 or 41, wherein the complex comprises a heterodimer of progranulin and prosaposin.

43. The cell culture according to any one of claim 6 or 10, further comprising a factor which stimulates secretion of progranulin, prosaposin or a heterodimer of progranulin and prosaposin from said cell line.

44. A device to treat a patient with a neurological disorder, comprising:
an implantable cell device; and
a cell line produced by the cell culture according to any one of claim 6 or 10, wherein said cell line is designed to secrete a therapeutic.

45. The device according to claim 44, wherein the implantable cell device comprises a capsule which contains said cell line.

46. The device according to claim 44, wherein the implantable cell device further comprises a semi-permeable membrane permitting the diffusion of said therapeutic secreted from said cell line situated within said implantable cell device through said membrane.

47. The device according to claim 46, wherein the semi-permeable membrane is immunoisolatory.

48. The device according to claim 46, wherein the device further comprises a matrix disposed within the semi-permeable membrane.

49. The device according to claim 44, further comprising a means to implant said cell device inside of a patient in need of treatment.

50. The device according to claim 49, wherein the implanting means comprises a catheter.

51. The device according to claim 50, wherein the catheter is designed to be implanted intrathecally into the striatum, spinal canal or into the subarachnoid space of the patient.

52. The device according to claim 44, further comprising one or more vehicles for delivery of therapeutics from the cell device.

53. The device according to claim 52, wherein the vehicles include a pump or syringe.

54. The device according to claim 44, wherein the device is implanted orally, intrathecally, intracerebroventricularly, or intracerebrally.

55. The device according to claim 44, wherein the neurological disorder is a neurodegenerative disease.

56. The device according to claim 44, wherein the neurological disorder is a lysosomal storage disease.

57. The device according to claim 55, wherein the neurodegenerative disease is selected from the group consisting of frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia, Parkinson's disease (PD), Multiple system atrophy (MSA) and lysosomal storage disorders.

58. The device according to claim 56, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease, Farber's disease, and combinations thereof.

59. A method for manufacturing a complex of progranulin and prosaposin, the method comprising inserting the steps of:
inserting a first expression construct which expresses progranulin into a cell line; and
inserting a second expression construct which expresses prosaposin into the same cell line.

60. The method according to claim 59, wherein the cell line is selected from the group consisting of: mouse myeloma cells (NS0), Chinese hamster ovary cells (CHO); Chinese hamster ovary cells (CHO)-K1; baby hamster kidney cells (BHK); mouse fibroblast-3T3 cells; African green monkey cell lines; mesenchymal chondroSarcoma-1 (MCS); rat adrenal pheochromocytoma (PC)-12; rat adrenal pheochromocytoma (PC)-12A; AT3, rat glial tumor (C6) cells; rat neuronal cell line RN33b; rat hippocampal cell line HiB5; growth factor expanded stem cells; epidermal growth factor (EGF)-responsive neurospheres; basic fibroblast growth factor (bFGF)-responsive neural progenitor stem cells derived from the central nervous system (CNS) of mammals; foetal cells; primary fibroblasts; Schwann cells; astrocytes; β-TC cells; Hep-G2 striatal cells; oligodendrocytes and their precursors; mouse myoblast cells-C2C12; human glial-derived cells-Hs683; human glial-derived cells-A172; HEI193T cell line; porcine glioblasts; neuronal cells; neurons; astrocytes; interneurons; chondroblasts isolated from human long bone; human embryonic kidney 293 cells (HEK293); human cell line HeLa cells; rabbit corneal-derived cells (Startus Seruminstitut Rabbit Conrnea cells (SIRC)); Human corneal derived cells, human choroid plexus cells, human induced pluripotent stem cells (iPS) derived cell lines, human neurotrophin 3 (NT3) cells, adult retinal pigments epithelial cell line-10 (ARPE-19), circulating angiogenic cells (CAC), immortalized human fibroblasts (MDX cells), telomerase immortalized human retinal pigment epithelium (RPE) cell lines and mesenchymal stem cells (MSC).

61. The cell culture according to claim 60, wherein the African green monkey cell lines are selected from the group consisting of COS-1, COS-7, SCC-1, BSC-40, BMT-10 and Vero cell lines.

62. The cell culture according to claim 60, wherein the human retinal pigment epithelium (RPE) cell line is human telomerase reverse transcriptase (hTERT) retinal pigment epithelium-1 (RPE-1).

63. The cell culture according to claim 60, wherein the preferred cell lines for mammalian recombinant production include ARPE-19, CHO, CHO-1, HEI193T, HEK293, COS, NS0, and BHK cells.

64. The method according to claim 59, wherein the first expression constructs comprises a plasmid.

65. The method according to claim 64, wherein the first expression construct further comprises a Sleeping beauty transposase system.

66. The method according to claim 59, wherein the second expression constructs comprises a plasmid.

67. The method according to claim 66, wherein the second expression construct further comprises a Sleeping beauty transposase system.

68. The method according to claim 59, wherein the cell line is contained in a bioreactor.

69. The method according to claim 59, further comprising the step of purifying the complex of progranulin and prosaposin.

70. The method according to claim 69, wherein the complex of progranulin and prosaposin is purified by ion exchange chromatography.

71. The method according to claim 70, wherein the ion exchange chromatography does not use polypropylene plastics.

72. The method according to claim 69, wherein the complex of progranulin and prosaposin is purified by gel filtration.

73. A therapeutic for the treatment of a neurological disorder, comprising a complex of progranulin and prosaposin according to the method of any one of claim 40 or 41.

74. The therapeutic according to claim 73, wherein said therapeutic is administered to a patient in need of treatment for a neurological disorder.

75. The therapeutic according to claim 74, wherein the neurological disorder is a neurodegenerative disease.

76. The therapeutic according to claim 74, wherein the neurological disorder is a lysosomal storage disease.

77. The therapeutic according to claim 57, wherein the neurodegenerative disease is selected from the group consisting of frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia, Parkinson's disease (PD) and Multiple system atrophy (MSA).

78. The therapeutic according to claim 76, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease and Farber's disease.

79. The therapeutic according to claim 73, wherein the therapeutic is administered to the patient by injection.

80. The therapeutic according to claim 73, wherein the therapeutic is administered to the patient by a catheter.

81. A method to test the relative concentrations of progranulin and prosaposin in a fluid sample from a patient, the method comprising the steps of:
testing for the concentration of progranulin;
testing for the concentration of prosaposin;
testing for the concentration of a complex of progranulin and prosaposin;
comparing the ratio of the concentration of progranulin to the concentration of a complex of progranulin and prosaposin; and
comparing the ratio of the concentration of prosaposin to the concentration of a complex of progranulin and prosaposin.

82. The method according to claim 81, wherein the test comprises an enzyme linked immunosorbent assay (ELISA) or proximity ligation assay.

83. The method according to claim 81, wherein the fluid sample from the patient is selected from the group consisting of human cerebrospinal fluid, plasma, serum, saliva, tear fluid, mother's milk, urine and combinations thereof.

84. The method according to claim 81, further comprising a step of diagnosing the patient with a neurological disorder.

85. The method according to claim 81, further comprising a step of assessing the patient's progression of a neurological disorder.

86. The method according to claim 84, wherein the neurological disorder is a neurodegenerative disease.

87. The method according to claim 84, wherein the neurological disorder is a lysosomal storage disease.

88. The method according to claim 86, wherein the neurodegenerative disease is selected from the group consisting of frontotemporal dementia (FTD), amyotrophic lateral sclerosis (ALS), Lewy body dementia, Parkinson's disease (PD), Gaucher's disease, neuronal ceroid lipofuscinosis, and combinations thereof.

89. The method according to claim 87, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease and Farber's disease.

90. An assay used to determine the absolute and relative levels of PGRN, PSAP and/or a PGRN/PSAP complex in a patient.

91. The assay according to claim 90, wherein said assay is used to diagnose a neurological disorder.

92. The assay according to claim 90, wherein said assay is used to assess a patient's progression of a neurological disorder.

93. The assay according to claim 90, wherein the neurological disorder is a neurodegenerative disease.

94. The assay according to claim 90, wherein the neurological disorder is a lysosomal storage disease.

95. The assay according to claim 93, wherein the neurodegenerative disease is selected from the group consisting of frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia, Parkinson's disease (PD) and Multiple system atrophy (MSA).

96. The assay according to claim 94, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease and Farber's disease.

97. A biomarker comprising a complex of progranulin and prosaposin.

98. The biomarker according to claim 97, wherein said biomarker is used to detect a neurological disorder and/or to assess the prognosis and progression of a neurological disorder.

99. The biomarker according to claim 98, wherein the neurological disorder is a neurodegenerative disease.

100. The biomarker according to claim 98, wherein the neurological disorder is a lysosomal storage disease.

101. The biomarker according to claim 99, wherein the neurodegenerative disease is selected from the group consisting of frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia, Parkinson's disease (PD) and Multiple system atrophy (MSA).

102. The biomarker according to claim 100, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease and Farber's disease.

103. The biomarker according to claim 97, wherein said biomarker is used to detect, diagnose and/or monitor an inflammatory disease, cancer and obesity-associated pathologies in a patient.

104. The biomarker according to claim 103, wherein said inflammatory disease is selected from the group consisting of cholelithiasis, fatty liver disease, endometriosis, inflammatory bowel disease, asthma, rheumatoid arthritis, chronic peptic ulcer, periodontitis, Crohn's disease, sinusitis, hepatitis, cardiovascular disease, arthritis, chronic obstructive pulmonary disease, encephalitis, meningitis, neuritis and pancreatitis.

105. The biomarker according to claim 103, wherein said obesity-associated pathologies are selected from the group consisting of Type 2 diabetes mellitus, Type 1 diabetes, hyperlipidemia, insulin insensitivity, hyperglycemia, hyperinsulinemia, hypoinsulinemia, dyslipidemia, hypertension and atherosclerosis.

106. A clonal cell culture, wherein said clonal cell culture expresses and releases a combination of factors.

107. The clonal cell culture according to claim 106, wherein said factor is a neurorestorative factor.

108. The clonal cell culture according to claim 106, wherein said factor is a lysosomal targeting factor.

109. The clonal cell culture according to claim 106, wherein said factor is a misfolded protein targeting factor.

110. The clonal cell culture according to claim 107, wherein said neurorestorative factor is selected from the group consisting of a neurotrophin protein, glial cell-derived neurotrophic factor protein, cerebral dopamine neurotrophic factor protein and mesencephalic astrocyte-derived neurotrophic factor protein.

111. The clonal cell culture according to claim 108, wherein said lysosomal targeting factor is selected from the group consisting of progranulin, a derivative of progranulin, prosaposin, a derivative of prosaposin, a progranulin/prosaposin complex, glucocerebrosidase, lysosomal-associated membrane protein 1 and cathepsin.

112. The clonal cell culture according to claim 109, wherein said misfolded protein targeting factor is a peptide, antibody or antibody fragment selected from the group consisting of alpha-synuclein, amyloid-beta (Aβ) tau, TAR DNA-binding protein 43, Fused in Sarcoma, Huntingtin protein and C9orf-derived dipeptide.

113. The clonal cell culture according to claim 109, wherein said misfolded protein targeting factor is conjugated to a functional peptide.

114. The clonal cell culture according to claim 113, wherein said functional peptide enhances cellular uptake.

115. The clonal cell culture according to claim 114, wherein said functional peptide is trans-activator of transcription (TAT) of the human immune-deficiency virus (HIV).

116. The clonal cell culture according to claim 113, wherein said functional peptide enhances triggers degradation pathways.

117. The clonal cell culture according to claim 116, wherein said functional peptide is proteolysis targeting chimera (PROTAC).

118. A combinatorial therapy administered to a patient in need thereof, wherein said therapy includes the delivery of progranulin, prosaposin, a complex of progranulin and prosaposin, alpha-synuclein targeting antibodies and a alpha-synuclein targeting neurorestorative factor.

119. The combinatorial therapy according to claim 118, wherein said progranulin, prosaposin, complex of progranulin and prosaposin, alpha-synuclein targeting antibodies and alpha-synuclein targeting neurorestorative factor are administered to the patient in different combinations.

120. The combinatorial therapy according to claim 118, wherein said alpha-synuclein targeting neurorestorative factor is glial cell-derived neurotrophic factor (GCNF).

121. The combinatorial therapy according to claim 118, wherein said combinational therapy treats neurological disorders.

122. The combinatorial therapy according to claim 121, wherein the neurological disorder is a neurodegenerative disease.

123. The combinatorial therapy according to claim 121, wherein the neurological disorder is a lysosomal storage disease.

124. The combinatorial therapy according to claim 122, wherein the neurodegenerative disease is selected from the group consisting of frontotemporal dementia (FTD), amyotrophic lateral sclerosis (ALS), Alzheimer's disease (AD), limbic-predominant age-related TAR DNA-binding protein-43 (TDP-43) encephalopathy (LATE), Lewy body dementia, Parkinson's disease (PD) and Multiple system atrophy (MSA).

125. The combinational therapy according to claim 123, wherein the lysosomal storage disease is selected from the group consisting of Gaucher's disease, atypical Gaucher's disease, metachromatic leukodystrophy, Krabbe disease, Kyoto encyclopedia of genes and genomes (KEGG) disease, neuronal ceroid lipofuscinosis (NCL), Mucopolysaccharidosis III and IV, Tay-Sachs disease and Farber's disease.

126. A cell line, wherein said cell line expresses or is modified to express a progranulin peptide, a prosaposin peptide or a complex of a progranulin peptide and a prosaposin peptide.
